(19) United States
(12) Patent Application Publication
(10) Pub. No.: US 2014/0287944 A1
(43) Pub. Date: Sep. 25, 2014

(54) MARKERS FOR FUNCTIONALLY MATURE BETA-CELLS AND METHODS OF USING THE SAME

(71) Applicant: President and Fellows of Harvard College, Cambridge, MA (US)

(72) Inventors: Sinisa Hrvatin, Cambridge, MA (US); Douglas A. Melton, Lexington, MA (US); Barak Blum, Chestnut Hill, MA (US)

(73) Assignee: President and Fellows of Harvard College, Cambridge, MA (US)

(21) Appl. No.: 14/191,057

(22) Filed: Feb. 26, 2014

Related U.S. Application Data
(60) Provisional application No. 61/769,614, filed on Feb. 26, 2013.
(60) Provisional application No. 61/789,488, filed on Mar. 15, 2013.

(51) Int. Cl. C12Q 1/68; G01N 33/50; G01N 33/74

(52) U.S. Cl. CPC C12Q 1/6881; G01N 33/74; G01N 33/507
USPC 506/9; 435/6.11; 435/7.1; 435/6.13; 435/7.92

(57) ABSTRACT

Markers for functionally mature β-cells and methods of using these markers are disclosed.

### CROSS-REFERENCE TO RELATED APPLICATIONS

This application claims the benefit of U.S. Provisional Application No. 61/769,614, filed Feb. 26, 2013, and U.S. Provisional Application No. 61/789,488, filed Mar. 15, 2013. The entire teachings of the above applications are incorporated herein by reference.

### GOVERNMENT SUPPORT

This invention was made with government support under U01 DK072473-07 awarded by the National Institutes of Health. The government has certain rights in the invention.

### BACKGROUND OF THE INVENTION

The directed differentiation of stem cells has the potential to produce β-cells for administration to individuals suffering from diseases associated with β-cell abnormality (e.g., diabetes). However, existing in vitro differentiation protocols often produce “β-like” cells, which do not have the same functional properties as mature β-cells. In addition, the complete set of signals and mechanisms governing β-cell maturation remains unknown.

### SUMMARY OF THE INVENTION

The present invention provides solutions to one or more of the problems outlined above. In particular, the present invention provides markers for identifying functionally mature β-cells and methods of using the markers for identifying mature β-cells, methods of identifying agents that modulate maturity of β-cells (e.g., agents that induce β-cell maturation to produce functional β-cells in vitro, or agents that induce β-cell maturation to produce functional β-cells in vivo), methods of modulating disorders associated with β-cell deficiency, and related compositions and methods.

In some aspects the present invention provides a method of determining the functional maturity of a β-cell or a population of β-cells, comprising: (a) obtaining a β-cell or a population of β-cells; (b) assaying the β-cell or population of β-cells for the presence or absence of one or more of: a GSIS response at low glucose concentrations, a large fold change in the GSIS response between low and high glucose concentrations, urocortin 3 (UCN3) protein, and elevated levels of UCN3 messenger ribonucleic acid (mRNA); and (c) determining the functional maturity of the β-cell or population of β-cells, wherein the β-cell or population of β-cells is: (i) functionally immature if the β-cell or β-cells in the population exhibit one or more of the presence of a GSIS response at low glucose concentrations, the absence of a large fold change in the GSIS response between low and high glucose concentrations, the absence of UCN3 protein, or the absence of elevated levels of UCN3 mRNA; or (ii) functionally mature if the β-cell or β-cells in the population exhibit one or more of the absence of a GSIS response at low glucose concentrations, the presence of a large fold change in the GSIS response between low and high glucose concentrations, the presence of UCN3 protein, or the presence of elevated levels of UCN3 mRNA.

In some embodiments the β-cell or population of cells are obtained from an in vitro source. In some embodiments the in vitro source is a culture of differentiating stem cells. In some embodiments the stem cells are selected from the group consisting of human embryonic stem cells (hESCs), induced pluripotent stem cells (iPSCs), blood stem cells, and combinations thereof. In some embodiments the in vitro source is selected from the group consisting of a cell bank, cell line, cell culture, cell population, and combinations thereof. In some embodiments the in vitro source is an ex-planted tissue or organ.

In some embodiments the β-cell is obtained from an in vivo source. In some embodiments the in vivo source is an individual who has received an administration of β-cells. In some embodiments the in vivo source is an individual suffering from a disorder associated with immature β-cells. In some embodiments the in vivo source is an individual suspected of being in need of functionally mature β-cells.

In some embodiments the presence of the GSIS response at low glucose concentrations is at least a first phase of insulin secretion in response to the low glucose concentration. In some embodiments the presence of the GSIS response at low glucose concentrations is a complete GSIS response comprising a first and second phase of insulin secretion in response to the low glucose concentration.

In some embodiments the absence of the GSIS response at low glucose concentrations is a lack of insulin secretion in response to the low glucose concentrations.

In some embodiments the low glucose concentration is less than or equal to about 5 mM. In some embodiments the low glucose concentration is about 2.8 mM.

In some embodiments the high glucose concentration is greater than or equal to about 10 mM. In some embodiments the high glucose concentration is about 16.7 mM.

In some embodiments the large fold change in the GSIS response between the low and high glucose concentrations is at least about 2.5 fold or 3.5 fold. In some embodiments the large fold change in the GSIS response between the low and high glucose concentrations is greater than or equal to about 50 fold.

In some embodiments assaying the β-cell or population of β-cells for the presence or absence of UCN3 protein comprises immunostaining.

In some embodiments assaying the β-cell or population of β-cells for elevated levels of UCN3 mRNA comprises conducting one or more hybridization assays. In some embodiments the one or more hybridization assays comprises using a microarray.

In some embodiments the presence of elevated levels of UCN3 mRNA comprises at least a 5 fold increase in the levels of UCN3 mRNA expression in the mature β-cells relative to the levels of UCN3 mRNA expression in the immature β-cells.

In some embodiments the method of determining the functional maturity of a β-cell or a population of β-cells further comprises sorting the functionally immature and mature β-cells identified in the population of β-cells. In some embodiments sorting the functionally immature and mature β-cells identified in the population of β-cells comprises fluorescence-activated cell sorting (FACS).

In some embodiments the method of determining the functional maturity of a β-cell or a population of β-cells further comprises quantifying the sorted functionally immature and mature β-cells identified in the population of β-cells.

In some embodiments the method of determining the functional maturity of a β-cell or a population of β-cells further comprises preserving the sorted functionally mature β-cells.

In some aspects the present invention provides a method of identifying an agent that modulates the functional maturity of β-cells, comprising: (a) contacting stem cells or differentiating β-cells with a test agent; (b) assaying the cells contacted with the test agent for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and (c) determining whether the test agent is a candidate agent that modulates the functional maturity of β-cells, wherein: (i) the test agent is a candidate agent that induces β-cells to become functionally immature if the β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNa; or (ii) the test agent is a candidate agent that induces β-cells to become functionally mature if the β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

In some embodiments the presence of elevated levels of UCN3 mRNA is assayed over a period of time during or after contact with the test agent.

In some embodiments the test agent is a combination of agents.

In some embodiments if the levels of UCN3 mRNA are increasing over the period of time during or after contact with the test agent, the cells are maturing into functionally mature β-cells.

In some embodiments the candidate agent is a candidate agent that modulates a disorder associated with immature β-cells. In some embodiments the disorder is diabetes. In some embodiments the disorder is prediabetes or hyperglycemia.

In some aspects the present invention provides a method of identifying the functional maturity status of an individual's β-cells, comprising: (a) obtaining a biological sample comprising β-cells from the individual; and (b) assaying the β-cells in the biological sample for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and (c) identifying the functional maturity of the individual's β-cells, wherein the individual's β-cells are: (i) functionally immature if the individual's β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNa; or (ii) functionally mature if the individual's β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA. If the individual's β-cells are functionally immature, the individual is in need of functionally mature β-cells.

In some embodiments the individual is a human or animal.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows that β-cell maturation is defined by a decrease in GSIS sensitivity to low glucose levels and by the expression of UCN3. (a) Three independent sets of 50 islets each, from P1 or P15 mice, were sequentially perfused with basal (0.5 mM, gray), low (2.8 mM, blue) or high (16.7 mM, red) glucose in a dynamic GSIS assay. Arrows indicate the time points at which solutions were changed. P1 islets display complete first and second phases of GSIS in response to low glucose, whereas P15 islets do not secrete insulin at this glucose concentration. (b) Triplicates of ten islets from P1 to adult were assayed for GSIS using low glucose (2.8 mM) and high glucose (16.7 mM). Two age groups can be distinguished according to their stimulation index (fold change in GSIS). ***, P<5×105. (c) Three independent sets of ten islets each from P1, P9 or P21 were assayed for GSIS using low glucose (2.8 mM, blue), high glucose (16.7 mM, red), 20 mM arginine (gray) or 30 mM KCl (green). The difference in the amount of insulin secreted between mature and immature islets is specific to glucose. *, P<0.05; **, P<0.001; NS, not significant). (d,e) Blood glucose (d) and insulin levels (e) in immature (P1, blue) and mature (P14, red) mouse pups. Insulin levels in the immature pups are higher than in the mature pups, although their blood glucose levels are lower. (f) Electron micrograph of insulin vesicles in β-cells at various ages. Scale bars, 2 βm. (g) Quantification of the number of insulin vesicles versus β-cell area of the data shown in f. (h) A scheme representing the microarray approach. Genes differentially expressed in both mature age groups compared to both immature age groups (i and ii) are chosen as candidate markers. (i) Representative scattered plot from the microarray. Note high similarity (R2) in gene expression between the mature (P10) and immature (P1) samples. (j) The expression levels of most β-cell markers are unchanged during GSIS maturation. Scatter plots of global gene expression from microarrays on FACS-sorted immature (P1) and mature (P10) β-cells. Red lines mark a twofold difference in expression and, with the exception of MafB, gene expression is not significantly different between these stages. (k) The expression of UCN3 mRNA at various ages as detected in the microarray. (l,m) Immunostaining of UCN3 (green) and insulin (red) on pancreata from E18.5 (l) and adult (m) mice. Nuclei are stained with DAPI (blue). Scale bars, 50 μm. Error bars, s.e.m. UCN3 is undetectable in E18.5 embryo. UCN3 is detected at high levels and co-localizes with adult β-cells.

FIG. 2 illustrates that UCN3 expression gradually increases during the course of mouse β-cell maturation in vivo and is expressed in hESC-derived β-like cells after differentiation and maturation in vivo, but not after the described differentiation in vitro. (a-c) Immunostaining of UCN3 (green) and insulin (red) on pancreata from P1, P6 and P22 mice. (d-f) Enlargement insets shown in a-c, respectively. Nuclei are stained with DAPI (blue). Scale bars, 50 μm. UCN3 in not detected at P1 even in large islets (a,d). At P6, some large islets express UCN3, but small aggregates do not express the peptide (arrows) (b,e). At P22, UCN3 is highly expressed in all islets (d,f). (g-i) Intracellular FACS analysis of insulin and UCN3 at 518.5, P6 and P13. Numbers in upper quadrants represent the percentage of insulin only (left) or insulin and UCN3 co-expressing cells (right) of all insulin-expressing cells (two upper quadrants), calculated as average±s.e.m. of three independent biological repeats (three separate litters) for each age group. (j) Experimental approach on hESCs differentiation. hESCs (ES, red) marked by Oct4 were differentiated in vitro into definitive endoderm (DE, yellow) marked by Sox17 and subsequently to pancreatic progenitors (PP, green), marked by the expression of Pdx1 and Nkx6.1. The cells were transplanted into SCID-beige mice to complete maturation in vivo. (k,l) Immunostaining for UCN3 (green) and insulin (red) on the in vitro differentiated cells shown at two magnifications (k, low magnification; l, high magnification). In vivo differentiated (transplanted) cells are shown in (m). Nuclei are stained with DAPI (blue). Scale bars, 50 μm. UCN3 is expressed in the in vivo matured cells, but not in in vitro differentiated, insulin-positive β-like cells.

FIG. 3 demonstrates that Ucn3 expression in mouse islets is restricted to β-cells. (A-C) Confocal images showing immunostaining of Ucn3 (green) and glucagon (red) on adult mouse pancreatic sections. (D-F) Ucn3 (green) and somatostatin (red) (G-I) Ucn3 (green) and pancreatic polypeptide (PPY, red). Nuclei are stained with DAPI (blue). Scale bars=50 μm. No co-localization of Ucn3 is seen with any of the islet hormones other than insulin.

FIG. 4 shows that Ucn3 expression levels increase gradually in all β-cells during maturation, whereas insulin content stays constant. Intra-cellular FACS analysis of insulin and Ucn3 in E18.5 (blue), P6 (green) and P13 (red). Histograms present the signal intensity of Ucn3 (A) and insulin (B) plotted against the percentage of all insulin expressing cells. Numbers in brackets show mean intensity±sem of three independent biological repeats (three separate litters) for each age group.

FIG. 5 shows UCN3 expression in human pancreas. (A-C) Confocal images showing immunostaining of UCN3 (green) and insulin (red) on pancreatic sections from an adult human. (D-F) UCN3 (green) and glucagon (red). (G-I) UCN3 (green) and somatostatin (red) (J-L) UCN3 (green) and pancreatic polypeptide (PPY, red). Nuclei are stained with DAPI (blue). Scale bars=50 μm.

FIG. 6 demonstrates that hESC-derived β-cells secrete human C-peptide in response to glucose challenge. Mice transplanted with 5 million hESC-derived pancreatic progenitors were fasted 12 weeks after transplantation overnight and injected with 2 mg/kg glucose. The levels of human C-peptide before (fasting, blue) and one hour after glucose administration (glucose, red) are shown. Despite variable basal levels of human C-peptide, all animals except mouse #4, showed glucose-stimulated secretion of human C-peptide.

FIG. 7 shows UCN3 expression in hESC-derived β-cells after maturation in vivo. (A-C) Confocal images showing immunostaining of UCN3 (green) and insulin (red) on hESC-derived graft 8 months post transplantation. (D-F) UCN3 (green) and glucagon (red). (G-I) UCN3 (green) and somatostatin (red) (J-L) UCN3 (green) and pancreatic polypeptide (PPY, red). Nuclei are stained with DAPI (blue). Scale bars=50 μm.

### DETAILED DESCRIPTION OF THE INVENTION

The present invention relates to novel markers for identifying the functional maturity of β-cells, and methods of using those markers for identifying functionally mature β-cells and distinguishing between immature and mature β-cells (e.g., determining whether an in vitro- or in vivo-differentiated β-cell has matured). In particular, the work described herein demonstrates that β-cell maturation is marked by an increased threshold for glucose stimulated insulin secretion (GSIS) and expression of the gene urocortin 3 (UCN3) (GenBank Gene ID: 114131, also known as SCP, SPC, UCNIII).

Accordingly, the present invention provides markers and methods for identifying the functional maturity of β-cells (e.g., distinguishing between immature and mature β-cells in a population), identifying agents that modulate the functional maturity of a β-cell or cause maturation/development of a stem cell or β-like cell to functional maturity, identifying agents that can modulate disorders associated with immature β-cells, identifying individuals in need of functionally mature β-cells, selecting functionally mature β-cells for administration to an individual (e.g., transplantation of mature β-cells into the individual, e.g., a human or animal), and identifying whether β-cells that have been administered to an individual or animal are mature β-cells in vivo.

In one aspect, the present invention provides a method of identifying a functionally mature β-cell. Generally, identifying the functional maturity of a β-cell or a population of β-cells can be accomplished by assaying the β-cell or population of β-cells for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA).

On one hand, if the β-cell or a population of β-cells exhibit one or more of the presence of a GSIS response at low glucose concentrations, the absence of a large fold change in the GSIS response between low and high glucose concentrations, the absence of UCN3 protein, or the absence of elevated levels of UCN3 mRNA, then the β-cell or a population of β-cells are functionally immature.

On the other hand, if the β-cell or a population of β-cells exhibit one or more of the absence of a GSIS response at low glucose concentrations, the presence of a large fold change in the GSIS response between low and high glucose concentrations, the presence of UCN3 protein, or the presence of elevated levels of UCN3 mRNA, then the β-cell or a population of β-cells are functionally mature.

Accordingly, in one aspect, the present invention provides a method of identifying the functional maturity of a β-cell or a population of β-cells. An exemplary method of identifying the functional maturity of a β-cell or a population of β-cells comprises: (a) obtaining a β-cell or a population of β-cells; and (b) assaying the β-cell or the population of β-cells for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and (c) identifying the functional maturity of the β-cell or the population of β-cells, wherein the β-cell or population of β-cells is: (i) functionally immature if the β-cell or β-cells in the population exhibit one or more of the presence of a GSIS response at low glucose concentrations, the absence of the large fold change in the GSIS response between low and high glucose concentrations, the absence of UCN3 protein, or the absence of elevated levels of UCN3 mRNA; or (ii) functionally mature if the β-cell or β-cells in the population exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

As used herein, “population of β-cells” includes a culture, cell line, cell bank, organ, or tissue, or a portion thereof, comprising β-cells. For example, a population of β-cells includes a whole pancreas or pancreatic tissue section, islets of Langerhans, or the like.

As used herein, a “functionally immature” β-cell refers to a cell that does not display one or more markers of β-cell functional maturity (e.g., UCN3, MAFB, NEUROD1, etc.) and lacks an appropriate GSIS response. For example, a functionally immature β-cell exhibits one or more of the presence of a GSIS response at low glucose concentrations, the absence of the large fold change in the GSIS response between low and high glucose concentrations, the absence of UCN3 protein, or the absence of elevated levels of UCN3 mRNA.

As used herein, a “functionally mature” β-cell refers to a cell that displays one or more markers of 13-cell functional maturity (e.g., UCN3, MAFB, NEUROD1, etc.) and exhibits an appropriate GSIS response. For example, a functionally mature β-cell exhibits one or more of the absence of a GSIS response at low glucose concentrations, the presence of the large fold change in the GSIS response between low and high glucose concentrations, the presence of UCN3 protein, or the presence of elevated levels of UCN3 mRNA.

The markers and methods of the present invention are capable of identifying the functional maturity of any β-cell or β-like cell. As used herein “β-like cell” refers to a cell that displays at least two markers indicative of a pancreatic β-cell, for example, expression of pancreas duodenum homeobox-1 (PDX-1), insulin, somatostatin, glucose transporter-2 (GLUT-2), glycogen, amylase, and neurogenin-3 (NGN-3). Markers indicative of pancreatic β-cells also include morphological characteristics (e.g., spherical shape), and insulin production and secretion. In a preferred embodiment a β-like cell is one which expresses insulin. B-like cells are not functionally mature β-cells.

In some embodiments of this and other aspects of the invention, the β-cell or population of β-cells is obtained from an in vitro source.

In some embodiments of this and other aspects of the invention, the in vitro source of β-cells is a culture of stem cells (e.g., differentiating stem cells). As used herein, “stem cell” refers to a cell that has the ability to differentiate into multiple cell types (e.g., pluripotent stem cells, totipotent stem cells, multipotent stem cells, blood stem cells, etc.). Examples of stem cells that can be used in the methods of the present invention include embryonic stem cells obtained by culturing a pre-implantation early embryo, embryonic stem cells obtained by culturing an early embryo prepared by somatic cell nuclear transfer, and induced pluripotent stem cells obtained by transferring appropriate transcription factors to a somatic cell to reprogram the cell. A variety of protocols for obtaining stem cells suitable for use in the methods of the present invention are available to the skilled artisan.

In some embodiments of this and other aspects of the invention, the stem cells are human embryonic stem cells (hESCs). In some embodiments of this and other aspects of the invention, the stem cells are induced pluripotent stem cells (iPSCs). In some embodiments of this and other aspects of the invention, the induced pluripotent stem cells are derived from reprogramming human somatic cells. The human somatic cells can be obtained from a healthy human or a human suffering from a disorder associated with immature or abnormal β-cells.

As used herein, “differentiated” β-cell or β-like cell refers to a β-cell or β-like cell obtained by differentiating a stem cell or other more naïve cell; such differentiating methods can comprise in vitro methods, in vivo methods or a combination of in vitro and in vivo methods. A “differentiating” β-cell refers to a cell (or cells) undergoing the process of differentiation. The present invention contemplates any culturing protocol that is capable of differentiating stem cells into β-cells or β-like cells. Examples of suitable protocols have been reviewed by Liew (Liew C G. Rev Diabet Stud 7(2), 82-92 (2010), incorporated herein by reference in its entirety.)

In some embodiments of this and other aspects of the invention, the in vitro source includes a cell bank (e.g., cryopreserved β-cells), a cell line, a cell culture (e.g., in vitro-differentiated β-cells), a cell population, and combinations thereof.

In some embodiments of this and other aspects of the invention, the in vitro source is an artificial tissue or organ (e.g., a pancreas, pancreatic islets, etc.).

In some embodiments of this and other aspects of the invention, the β-cell is obtained from an in vivo source.

In some embodiments of this and other aspects of the invention, the in vivo source is an individual or animal who has received an administration of β-cells. In such embodiments, an individual or animal can be administered functionally mature β-cells (e.g., via transplantation) and the markers and methods of the present invention can be used to confirm that the administered β-cells remain mature post-administration. Alternatively, an individual or animal can be administered functionally immature β-cells (e.g., in vitro-differentiated insulin positive β-like cells), and the markers and methods of the present invention can be used to determine whether the functionally immature β-cells have matured in vivo.

In some embodiments of this and other aspects of the invention, the in vivo source is an individual suffering from a disorder associated with immature β-cells (e.g., prediabetes or diabetes).

In some embodiments of this and other aspects of the invention, the in vivo source is an individual suspected of being in need of functionally mature β-cells. In such embodiments, the methods of identifying the functional maturity of β-cells can be adapted for use in methods of identifying individuals in need of functionally mature β-cells. For example, a biological sample comprising β-cells can be obtained from the individual, and the β-cells in the biological sample can be assessed for their maturity in accordance with the methods of the present invention.

In some embodiments of this and other aspects of the invention, the in vivo source is a tissue or organ obtained from a donor individual. In such embodiments, the markers or methods of the present invention can be used to determine whether the β-cells in the tissue or organ (e.g., pancreas, islets of Langerhans, etc.) are functionally mature before transplanting the tissue or organ into the recipient individual.

Typically, assaying a β-cell or population of β-cells for the presence or absence of a GSIS response at low glucose concentrations and/or for the presence or absence of a large fold change in the GSIS response between the low and high glucose concentrations involves conducting a GSIS assay. Briefly, a GSIS assay involves exposing a β-cell or population of β-cells to varying concentrations of glucose and measuring how much insulin is secreted by the β-cell or population of β-cells in response to the varying glucose concentrations.

The present invention contemplates the use of any method of measuring insulin secretion available to the skilled artisan. An exemplary method of measuring insulin secretion from isolated islets of Langerhans is described by Nolan and O-Dowd (Methods Mol Biol 560, 43-51 (2009)). Other suitable methods of measuring insulin secretion are apparent to the skilled artisan.

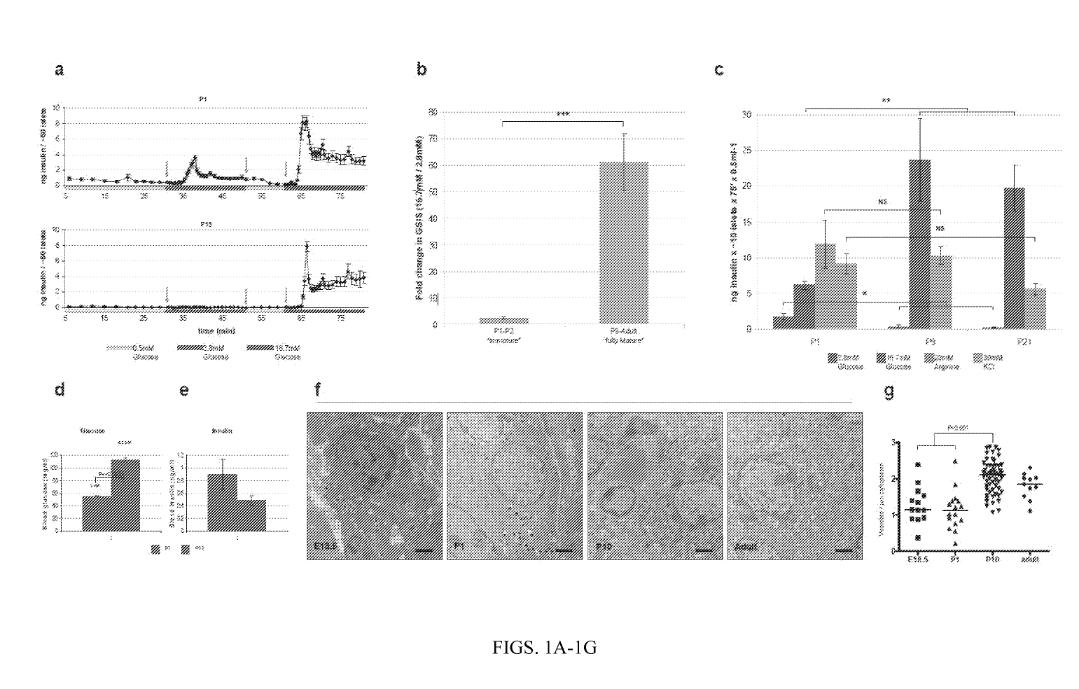

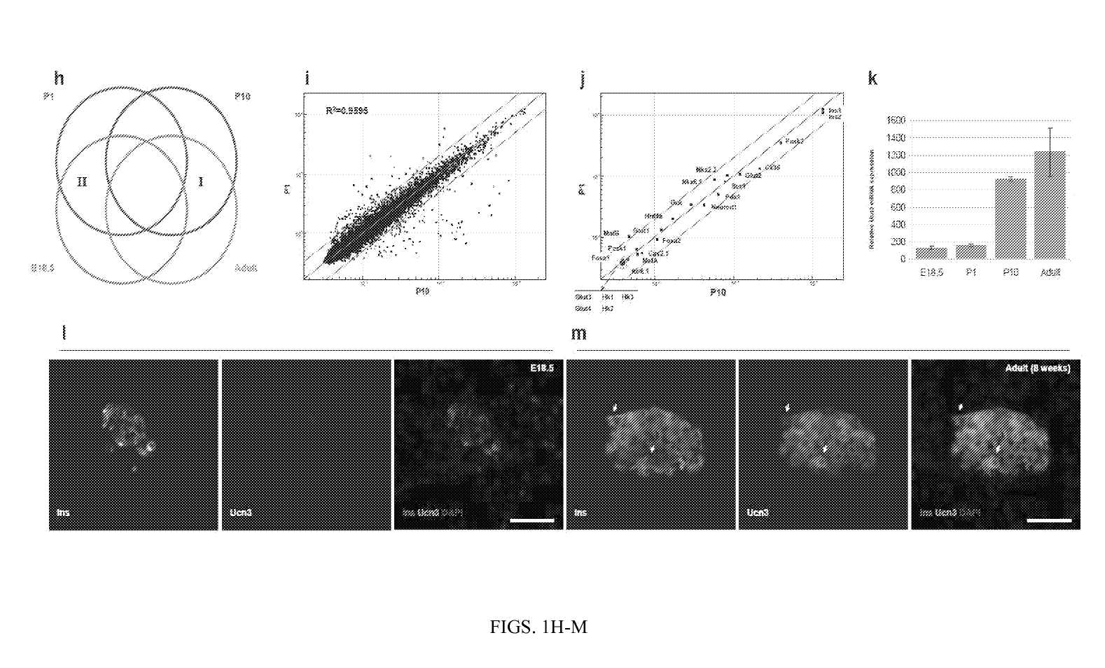

As noted above, the presence of a GSIS response at low glucose concentrations is indicative of immature β-cells. As used herein, “presence of a GSIS response at low glucose concentrations” generally means that a statistically measurable and relevant amount of insulin is secreted by the cells upon exposure to low concentrations of glucose. In some embodiments of this and other aspects of the invention, the presence of a GSIS response at low glucose concentrations is at least a first phase of insulin secretion in response to the low glucose concentration. In some embodiments of this and other aspects of the invention, the presence of a GSIS response at low glucose concentrations is a complete GSIS response comprising a first and second phase of insulin secretion in response to the low glucose concentration. FIG. 1a illustrates an example of a complete GSIS response.

Conversely, the absence of a GSIS response at low glucose concentrations is indicative of mature β-cells. In some embodiments of this and other aspects of the invention, the absence of a GSIS response at low glucose concentrations is a lack of insulin secretion in response to the low glucose concentrations.

As used herein, a “low glucose concentration” refers to concentrations of glucose that are less than or equal to about 5 mM. In some embodiments of this and other aspects of the invention, the low glucose concentration is between about 2.8 mM and about 5 mM. In some embodiments of this and other aspects of the invention, the low glucose concentration is about 2.8 mM. In some embodiments of this and other aspects of the invention, the low glucose concentration is below 2.8 mM. In some embodiments of this and other aspects of the invention, the low glucose concentration is about 0.5 mM.

As used herein, a “high glucose concentration” refers to concentrations of glucose that are greater than or equal to about 10 mM. In some embodiments of this and other aspects of the invention, the high glucose concentration is about 16.7 mM. In some embodiments of this and other aspects of the invention, the high glucose concentration is about 20 mM or more.

In accordance with the present invention, the presence or absence of a large fold change in the GSIS response of a β-cell between exposure to low and high glucose concentrations is a marker for β-cell functional maturity. For example, the absence of a large fold change in the GSIS response between the low and high glucose concentrations is indicative of functionally immature β-cells. In contrast, the presence of a large fold change in the GSIS response between the low and high glucose concentrations is indicative of functionally mature β-cells. FIG. 1b illustrates an exemplary large fold change in the GSIS response between the low and high glucose concentrations. Of course, those skilled in the art will appreciate that the large fold change may vary depending on a variety of factors (e.g., the low and high) concentrations of glucose used in the GSIS assay, the extent to which the β-cells are functionally mature, etc.).

In some embodiments of this and other aspects of the invention, the large fold change in the GSIS response between the low and high glucose concentrations is at least about 2.5 fold. In some embodiments of this and other aspects of the invention, the large fold change in the GSIS response between the low and high glucose concentrations is at least about 3.5 fold. In some embodiments of this and other aspects of the invention, the large fold change in the GSIS response between the low and high glucose concentrations is about 5 fold, 10 fold, 15, fold, 20, fold, 25, fold, 28 fold, 32 fold, 36 fold, 39 fold, 41 fold, 43 fold, 45 fold, up to 47 fold or more. In some embodiments of this and other aspects of the invention, the large fold change in the GSIS response between the low and high glucose concentrations is at least about 50 fold. In some embodiments of this and other aspects of the invention, the large fold change in the GSIS response between the low and high glucose concentrations is at about 50 fold, about 55 fold, about 60 fold, about 70 fold, or up to about 75 fold, or more.

According to some aspects of the present invention, the presence or absence of UCN3 protein production is a marker for β-cell functional maturity. For example, the absence of UCN3 protein is indicative that the β-cell or population of β-cells is functionally immature, while the presence of UCN3 protein is indicative that the β-cell or population of β-cells is functionally mature. FIG. 11 and FIG. 1m illustrate an exemplary use of UCN3 protein as a marker for β-cell functional maturity.

The present invention contemplates detecting the presence or absence of UCN3 protein according to any technique available to the skilled artisan. In some embodiments of this and other aspects of the invention, assaying for the presence or absence of UCN3 protein comprises immunostaining (e.g., Western blotting, immunohistochemistry, ELISA, etc). In such embodiments, anti-UCN3 antibodies targeted to UCN3 protein (or a portion, variant, or fragment thereof) are used to detect the presence or absence of UCN3 protein.

Such antibodies can include polyclonal antibodies, monoclonal antibodies, chimeric antibodies, single-chain antibodies, antibody fragments, humanized antibodies, multi-specific antibodies, and modified antibodies (e.g., fused to a protein to facilitate detection.) Suitable anti-UCN3 antibodies can be generated according to routine protocols, or can be readily obtained from a variety of commercial sources (e.g., Sigma-Aldrich). Other suitable techniques for detecting the presence of UCN3 protein in β-cells are apparent to those skilled in the art.

According to other aspects of the present invention, the presence or absence of elevated levels of UCN3 mRNA in a β-cell is a marker for β-cell functional maturity. For example, the absence of elevated levels of UCN3 mRNA in a β-cell or population of β-cells is indicative that the β-cell or population of β-cells is functionally immature, while the presence of elevated levels of UCN3 mRNA in a β-cell or population of β-cells is indicative that the β-cell or population of β-cells is functionally mature. FIG. 1k illustrates an exemplary use of elevated levels of UCN3 mRNA as a marker for β-cell functional maturity.

It is to be understood that the phrase “elevated levels of UCN3 mRNA” refers to levels of UCN3 mRNA in a mature β-cell relative to an immature β-cell. That is, levels of UCN3 mRNA are higher in mature β-cells relative to levels of UCN3 mRNA in immature β-cells, which are lower. In this regard, it should also be appreciated that a maturity gradient of elevated UCN3 mRNA levels exists between UCN3 mRNA levels that are not elevated (i.e., immature β-cells), elevating UCN3 mRNA levels (i.e., maturing β-cells), and elevated UCN3 mRNA levels (i.e., mature β-cells). In other words, there is a gradual increase in UCN3 mRNA levels as β-cells mature.

The present invention contemplates detecting the presence or absence of elevated levels of UCN3 mRNA according to any technique available to the skilled artisan. In some embodiments of this and other aspects of the invention, assaying the β-cell or population of β-cells for elevated levels of UCN3 mRNA comprises conducting one or more hybridization assays. In some embodiments of this and other aspects of the invention, the one or more hybridization assays comprises a microarray.

In some embodiments of this and other aspects of the invention, the presence of elevated levels of UCN3 mRNA comprises at least a 2 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 3 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 4 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 5 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 6 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 7 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 8 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 9 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises at least a 10 fold increase in the levels of UCN3 mRNA in the mature β-cells relative to the levels of UCN3 mRNA expression in the immature β-cells. In some embodiments of this and other aspects of the invention, the presence of elevated levels of UCN3 mRNA comprises between about a 5 fold increase and about a 10 fold increase in the levels of UCN3 mRNA in the mature β-cells relative to the levels of UCN3 mRNA expression in the immature β-cells.

In some embodiments of this and other aspects of the invention, the present invention contemplates sorting functionally immature and mature β-cells identified in a population of β-cells. Sorting immature and mature β-cells can be helpful for selecting mature β-cells useful for administration to an individual in need of mature β-cells. Suitable methods of sorting cells will be apparent to the skilled artisan. In some embodiments of this and other aspects of the invention, sorting the functionally immature and mature β-cells identified in the population of β-cells is achieved by fluorescence-activated cell sorting (FACS).

It should be appreciated that FACS analysis can be performed in combination with the methods for detecting UCN3 expression to sort β-cells expressing certain markers and quantify the percentage and levels of UCN3 expression and insulin production, as well as to analyze global gene expression patterns.

In some embodiments of this and other aspects of the invention, the present invention contemplates quantifying the sorted functionally immature and mature β-cells identified in the population of β-cells.

In some embodiments of this and other aspects of the invention, the present invention contemplates preserving the sorted functionally mature β-cells (e.g., cryopreservation of the cells in appropriate reagents).

The markers of the present invention can be measured in β-cells or populations of β-cells to assay for agents that modulate β-cell maturity (e.g., agents that induce β-cells to mature into functionally mature β-cells or agents that induce mature β-cells to become functionally immature β-cells). Identification of one or more agents (or factors) that induce functional β-cell maturation can be used for the in vitro production of functionally mature β-cells for administration to a human or animal in need of such functionally mature β-cells (e.g., an individual suffering from a disorder associated with immature β-cells, e.g., prediabetes or diabetes). Identification of agents (or factors) that induce mature β-cells to dedifferentiate into functionally immature β-cells can be used to understand mechanisms underlying disorders associated with immature β-cells, as well as to identify conditions in culture which might need to be inhibited to produce functionally mature β-cells in vitro.

Accordingly, in another aspect, the present invention provides a method of identifying an agent that modulates the functional maturity of β-cells. An exemplary method of identifying an agent that modulates the functional maturity of β-cells comprises: (a) contacting stem cells or β-like cells or β-cells with a test agent; (b) assaying the cells contacted with the test agent for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and (c) identifying the test agent as a candidate agent that modulates the functional maturity of β-cells, wherein: (i) the test agent is a candidate agent that induces β-cells to become functionally immature if the β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNA; or (ii) the test agent is a candidate agent that induces β-cells to become functionally mature if the β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

In some embodiments of this and other aspects of the invention, the presence of elevated levels of UCN3 mRNA is assayed over a period of time during or after contact with the test agent. In such embodiments, if the levels of UCN3 mRNA are increasing over the period of time during or after contact with the test agent, the β-cells are maturing into functionally mature β-cells.

It should be appreciated that candidate β-cell maturity modulating agents identified according to the methods of the invention may be used in methods of treating disorders associated with immature β-cells. For example, an agent that induces immature β-cells to become functionally mature β-cells can be used to treat a disorder associated with immature β-cells. Accordingly, in some embodiments of this and other aspects of the invention, the candidate agent is a candidate agent that modulates a disorder associated with immature β-cells. In some embodiments of this and other aspects of the invention, the disorder is diabetes. In some embodiments of this and other aspects of the invention, the disorder is prediabetes or hyperglycemia.

Those skilled in the art will appreciate how to perform the identification methods (e.g., identifying agents for modulating β-cell maturity, identifying agents that modulate disorders associated with immature β-cells, etc.) of the present invention using routine protocols available to the skilled artisan (e.g., high-throughput screening, combinatorial chemistry, in silico screening, etc.).

It should be appreciated that a wide variety of test agents can be used in the methods (e.g., small molecules, polypeptides, peptides, nucleic acids, oligonucleotides, lipids, carbohydrates, or hybrid molecules).

In another aspect, the present invention provides a method of identifying the functional maturity of an individual's β-cells. An exemplary method of identifying the functional maturity of an individual's β-cells comprises: (a) obtaining a biological sample comprising β-cells from the individual; (b) assaying the β-cells in the biological sample for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and (c) identifying the functional maturity of the individual's β-cells, wherein the individual's β-cells are: (i) functionally immature if the individual's β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNa; or (ii) functionally mature if the individual's β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

The present invention contemplates obtaining a biological sample comprising β-cells from the individual according to any technique available to the skilled artisan. The individual from whom the biological sample is obtained may be a healthy individual, or an individual suffering from a disorder associated with functionally immature β-cells.

In some embodiments of this and other aspects of the invention, if the individual's β-cells are functionally immature, the individual is in need of functionally mature β-cells.

In some embodiments of this and other aspects of the invention, the individual is a human or animal.

In some embodiments of this and other aspects of the invention, the functional maturity of the individual's β-cells is identified before β-cells are administered to the individual. In some embodiments of this and other aspects of the invention, the functional maturity of the individual's β-cells is identified after β-cells have been administered to the individual.

In some embodiments of this and other aspects of the invention, the biological sample comprises pancreatic tissue. In some embodiments of this and other aspects of the invention, the biological sample comprises islets of Langerhans.

The markers of the present invention can be used for selecting functionally mature β-cells for administration to a human or animal subject in need of such functionally mature β-cells, as well as determining whether in vitro β-like cells administered to a human or animal subject are functionally mature in vivo.

Accordingly, the present invention provides methods of determining whether in vitro differentiated β-like cells administered to a human or animal subject are functionally mature in vivo. An exemplary method of determining whether in vitro-differentiated β-like cells administered to a human or animal subject are functionally mature in vivo comprises (a) providing in vitro-differentiated β-like cells, (b) administering the in vitro-differentiated β-like cells to the human or animal subject, (c) obtaining a biological sample comprising β-cells from the human or animal subject, (d) assaying the β-cells in the biological sample for an increased threshold for GSIS, UCN3 expression, or both an increased threshold for GSIS and UCN3 expression, and (e) determining that the in vitro-differentiated β-like cells administered to the human or animal subject are functionally mature in vivo if the β-cells in the biological sample exhibit the increased threshold for GSIS, exhibit UCN3 expression or exhibit both the increased threshold for GSIS and UCN3 expression.

In some embodiments of this and other aspects of the invention, the β-like cells are insulin-positive β-like cells. In some embodiments of this and other aspects of the invention, the insulin-positive β-like cells comprise Pdx+ and/or Nkx6.1+ pancreatic progenitor cells.

In some embodiments of this and other aspects of the invention, administering the in vitro-differentiated β-like cells to the human or animal subject comprises transplanting the β-like cells to the human or animal subject (e.g., into a kidney capsule of the human or animal subject). Other suitable methods of administering the in vitro-differentiated β-like cells to the human or animal subject are apparent to the skilled artisan.

In some instances, it may be desirable to conduct a glucose tolerance test on the human or animal subject to which the β-like cells have been administered to detect levels of human fasting C-peptide in the human or animal subject. It should be appreciated that if the levels of fasting human C-peptide levels detected are above a background level after administration of the β-like cells to the human or animal subject, the administered β-like cells are functionally mature in vivo. In this way, fasting human C-peptide levels can be used as an additional marker to confirm that the administered β-like cells have functionally matured in vivo (e.g., the functionally mature β-like cells are glucose-responsive β-cells).

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### Functional β-Cell Maturation is Marked by an Increased Glucose Threshold and by Expression of Urocortin 3 (UCN3)

### Materials and Methods

### Animal Experiments and Islet Isolation

All animal experiments were performed in compliance with the Harvard University International Animal Care and Use Committee (IACUC) guidelines. Mouse strains used in this study were ICR, Swiss-Webster wild type and Pdx1-GFP transgenic and SCIDbeige mice. Blood glucose levels were measured using OneTouch Ultra2 glucometer (LifeScan). Blood insulin levels were measured with an Ultrasensitive Insulin ELISA kit (Alpco). For glucose tolerance test, animals were fasted over-night and blood was taken from tail tips before, and 1 hour after, injection of 2 gr/kg body weight glucose. Human C-peptide levels were measured using Human C-peptide ELISA kit (Alpco). For islet isolation, adult pancreata were perfused through the common bile duct with 0.8 mM Collagenase P (Roche) and fetal and neonatal pancreata were dissected wholly without perfusion. Pancreata were digested with 0.8 mM Collagenase P (Roche) and purified by centrifugation in Histopaque gradient (Sigma).

### Glucose Stimulated Insulin Secretion (GSIS) Assays

Isolated islets were recovered over night in islet media (DMEM containing 1 gr/L glucose, 10% v/v FBS, 0.1% v/v Penicillin/Streptomycin). Islets were picked manually under a fluorescent dissecting microscope according to their GFP fluorescence. Care was taken to pick islets of approximately the same size from all ages. For dynamic GSIS, approximately 50 islets were hand picked and assayed on a fully automated Perifusion System (BioRep). Chambers were sequentially perfused with 0.5 mM, 2.8 mM or 16.7 mM glucose in KRB buffer (128 mM NaCl, 5 mM KCl, 2.7 mM CaCl2, 1.2 mM MgCl2, 1 mM Na2HPO4, 1.2 mM KH2PO4, 5 mM NaHCO3 HEPES, 0.1% BSA) at a flow rate of 0.1 ml/min. Fractions were collected and kept at −80° C. until analysis. For static GSIS assays, approximately 10 islets were handpicked, incubated for 2 hours in KRB buffer at 37° C., 5% CO2, and then incubated for 75 min with 2.8 mM or 16.7 mM glucose in the same conditions. Insulin concentrations in the supernatant were determined using Ultrasensitive Insulin ELISA kit (Alpco). Analysis of the results was done using Matlab software.

### Electron Microscopy

Samples were fixed with 4% paraformaldehyde and 0.1% glutaraldehyde for 2 h at room temperature and further refixed with a mixture of 1% osmiumtetroxide (OsO4) plus 1.5% potassium ferrocyanide (KFeCN6) for 2 h, washed in water and stained in 1% aqueous uranyl acetate for 1 h followed by dehydration in grades of alcohol (50%, 70%, 95%, 2×100%) and propyleneoxide (1 h). Samples were then infiltrated in propyleneoxide:Epon 1:1 overnight and embedded in TAAB Epon (Marivac Canada Inc.). Ultrathin sections (about 60-80 nm) were cut on a Reichert Ultracut-S microtome, picked up on to copper grids, stained with 0.2% lead citrate and examined in a Tecnai G2 Spirit BioTWIN transmission electron microscope. Images were taken with an AMT CCD camera. The number of insulin vesicles and cell area were determined using ImageJ software.

### Microarray Analysis

Islets were isolated as above from heterozygous Pdx1-GFP (crossed with ICR) animals and further dissociated into single cells with 0.25% Trypsin-EDTA (Invitrogen). GFP+ cells were isolated using FACSaria (BD Bioscience). Total RNA was extracted with RNeasy RNA extraction kit (Qiagen). Biotinylated cRNA was prepared from ≧100 ng of isolated RNA using Illumina TotalPrep RNA Amplification Kit (Ambion) and hybridized to the Illumina mouse genome Bead Chips (MouseRef8). All samples were prepared as four biological replicates. Data were acquired with Illumina Beadstation 500 and were evaluated using BeadStudio Data Analysis Software (Illumina).

### Immunohistochemistry and FACS Analyses

For immunohistochemistry, pancreata were fixed by immersion in 4% paraformaldehyde overnight at 4° C.

Samples were washed with PBS, incubated in 30% sucrose solution overnight and embedded with optimal cutting temperature compound (Tissue-Tek). 10 μm sections were blocked with 10% donkey serum (Jackson Immunoresearch) in PBS/0.1% Triton X and incubated with primary antibodies overnight at 4° C. Secondary antibodies were incubated for 1 hr at room temperature. The following primary antibodies and dilutions were used: rabbit anti-mouse or anti-human Ucn3 (1:600-1:800, both from Phoenix Pharmaceuticals), rabbit anti-human Ucn3 (1:600, a gift from Dr. Wylie Vale, Salk Institute), Guinea Pig anti-insulin (1:800, DAKO), Guinea Pig antiglucagon (1:200, Linco), Goat anti-Somatostatin (1:200, Santa Cruz) and Goat anti-PPY (1:200, Novus). Secondary antibodies were: Alexa Fluor 488 donkey anti-rabbit (1:400, Invitrogen), Alexa Fluor 647 donkey anti-goat (1:400, Invitrogen) and DyLight 649 donkey anti-guinea pig (1:400, Jackson Immunoresearch). Nuclei were visualized with DAPI. It is important to note that, in our hands, anti-human immunostaining was successful only on human tissues fixed over-night with 4% paraformaldehyde directly after surgery. Efforts to use the abovementioned anti-human Ucn3 antibodies on flashfrozen-unfixed cryosections, acetone-fixed cryosections or formalin-fixed-paraffinembedded samples resulted in either close-to-background or non-specific staining.

Images were taken using an Olympus IX51 Microscope or Zeiss LSC 700 confocal microscope. For intra-cellular FACS analysis, islets were isolated as above and further dissociated into single cells with 0.25% Trypsin-EDTA (Invitrogen). The cells were then fixed with Cytofix/Cytoprem solution (BD Biosciences) at 4° C. for 30 min, washed once with Perm Wash Buffer (BD Biosciences), and stained with Guinea Pig anti-insulin (1:800, DAKO) and rabbit anti-Ucn3 (1:600, Phoenix Pharmaceuticals) for 1 hour at room temperature. The cell were then washed once with Perm Wash Buffer (BD Biosciences), incubated with TexasRed donkey anti-guinea pig (1:400, Jackson Immunoresearch) and Alexa Fluor 488 donkey anti-rabbit (1:400, Invitrogen) for 45 min at room temperature, washed with PBS, filtered through a nylon mash, and analyzed LSR-II FACS machine (BD Biosciences). Analysis of the results was done using FlowJo software.

### HESC Culture and Differentiation

Human ESCs (WA1) were cultured on Matrigel (BD Biosciences) in mouse embryonic fibroblast conditioned media (MEF-CM). MEF-CM media was produced by conditioning media for 24 days on a confluent layer of mouse embryonic fibroblasts and subsequently adding 20 ng/ml bFGF (Invitrogen). The media was composed of DMEM/F12 (GIBCO) media supplemented with 20% KnockOut Serum Replacement (GIBCO), 2 mM Lglutamine (L-Glu, GIBCO), 1.1 mM 2-mercaptoethanol (GIBCO), 1 mM nonessential amino acids (GIBCO), 1× penicillin/streptomycin (GIBCO). Cells were passaged at the ratio of 1:6-1:20 every 4-7 days using TrypLE Express (Invitroge). To initiate differentiation, the cells were cultured as previously described 1 onto 1:30 dilution of growth factor reduced matrigel (BD Biosciences) in MEF-CM. Two to three days following seeding the differentiation was initiated as follows: cells were exposed to RPMI 1640 (Invitrogen) supplemented with 0.2% fetal bovine serum (FBS) (Hyclone, Utah), 100 ng/mL activin-A (AA; Pepro-tech; Rocky Hill, N.J.), and 20 ng/mL of Wnt3A (R&D Systems) for day one only. For days 2-3, cells were cultured in RPMI with 0.5% FBS and 100 ng/mL AA (stage 1). During days 4-5 cells were treated with DMEM-F12 medium containing 2% FBS and 50 ng/ml FGF7 (Peprotech) (stage 2). For days 6-9 cells were treated with DMEM-HG (Invitrogen), 1% (v/v) B27 (Invitrogen), 2 uM RA (Sigma), 0.25 uM SANT-1 (Sigma), and 100 ng/ml rhNoggin (R&D Systems) (stage 3).

During days 10-13 cells were treated with DMEM-HG Invitrogen)+1% (v/v) B27 (Invitrogen), 100 ng/ml rhNoggin (R&D Systems), 50 nM TPB (PKC activator, EMD Biosciences), and 1 μM ALK5 inhibitor II (Axxora, San Diego, Calif.) (stage 4). On day 14, cells were treated with 5 mg/mL Dispase for 5 min, followed by gentle pipetting to mix and break the cell clumps into small clusters (<100 micron). The cell clusters were cultured for one day in a 125 ml Spinner Flask (Corning) at 80-100 rpm overnight with DMEM-HG supplemented with 1 μM ALK5 inhibitor II, 100 ng/mL of Noggin and 1% B27.

For transplantation into mice, 10 million cells in clusters were transplanted under the kidney capsule of SCID-Bg mice (Jackson Laboratory). 8 months following transplant the graft was surgically extracted from under the mouse kidney capsule, fixed in 4% paraformaldehyde (PFA, Sigma), equilibrated in 30% sucrose, embedded in O.C.T., cryopreserved and sectioned.

The directed differentiation of human pluripotent stem cells (hPSCs) has the potential to produce β-cells for transplantation into diabetics. However, the available protocols for in vitro differentiation produce only “β-like” cells. These β-like cells do not perform the accurate glucose-stimulated insulin secretion (GSIS) found in mature β-cells unless they are transplanted into mice and allowed to further differentiate for many weeks (Kroon et al. Nat. Biotechnol. 26, 443-452 (2008)). During normal development, insulin-expressing β-cells appear around embryonic day 13.5 (E13.5) in mice or week 8 and 9 post-conception in humans (Pan and Wright. Dev. Dyn. 240, 530-565 (2011); Slack, J. M. Development 121, 1569-1580 (1995)), but regulated GSIS has been observed only days after birth. The signals and mechanisms governing β-cell maturation, either during postnatal development or after transplantation, are unknown.

During the course of work described herein, the present inventors investigated functional β-cell maturation based on glucose GSIS parameters, and identified markers of functionally mature β-cells that can be used to make functional stem cell-derived (e.g., hPSC) β-cells in culture. GSIS is typically measured by the fold change in insulin secretion between low (2.8-5 mM) and high (>10 mM) glucose concentrations (Rozzo et al. NY Acad. Sci. 1152, 53-62 (2009)). In this assay, it was observed that neonatal β-cells displayed a high basal insulin secretion at low glucose concentrations, and stimulation with a high concentration of glucose resulted in a small fold-increase in insulin secretion, suggesting that either neonatal β-cells have uncontrolled insulin ‘leakiness’ at low glucose concentrations, or alternatively, they have a lower glucose concentration threshold at which they secrete insulin.

During the course of work described herein, the present inventors conducted dynamic GSIS assays on neonatal (1 d old, P1) and older (15 d old, P15) mouse islets using a very low baseline glucose level of 0.5 mM. Neonatal P1 islets execute a complete GSIS response (both first and second phases of insulin secretion) at low (2.8 mM) glucose concentrations, whereas P15 islets have no response (no insulin secretion) at this concentration (FIG. 1a). These results demonstrate that immature β-cells are not leaky, but rather have a lower threshold for GSIS, secreting insulin in response to a lower glucose concentration than mature β-cells.

During the course of work described herein, the present inventors investigated when β-cells acquire a mature GSIS capacity, and tested mouse islets isolated from P1 to adult for their response to low (2.8 mM) and high (16.7 mM) glucose concentrations. Islets from neonatal mice, ages P1 and P2, secreted 2.6-±0.5-fold more insulin in high glucose than in low glucose, respectively, whereas islets from P9 to adult secreted, on average, 60.9-±10.7-fold more insulin in high glucose than in low glucose, respectively (FIG. 1b). Thus, the large change in GSIS response between low and high glucose that characterizes β-cell maturation occurs between P2 and P9. Islets of mice younger than P2 display an ‘immature’ response, whereas islets from mice older than P9 respond as ‘mature’ β-cells. From P3 to P8, a mixed (intermediate) GSIS phenotype was observed.

Notably, the difference in insulin secretion between mature and immature β-cells is specific for glucose. The amount of insulin secreted by P1 and P9 islets in response to 20 mM arginine was 11.9±3.5 ng and 10.3±1.1 ng, respectively. The amount of insulin secreted from P1 and P21 islets in response to 30 mM KCl was 9.17±1.4 ng and 5.66±0.9 ng, respectively. These differences are not statistically significant (FIG. 1c). In contrast, islets from P1 mice secreted only 6.2±0.6 ng insulin, during 75 min in 0.5 ml high (16.7 mM) glucose, whereas the same number of islets from P9 and P21 mice secreted 23.7±5.7 ng and 19.7±3.2 ng insulin, respectively (difference between P1 and older mice, P<0.001). At low glucose levels, the opposite trend was observed: P1 islets secrete 1.8±0.5 ng insulin at 2.8 mM glucose, whereas P9 and P21 islets secrete only 0.4±0.3 ng and 0.3±0.1 ng insulin, respectively (P<0.05) (FIG. 1c).

During the course of work described herein, the present inventors investigated the physiological consequences in vivo of the differences observed in vitro between mature and immature β-cells' response to glucose. Consistent with previous reports (Rozzo et al. 2009), P1 pups had significantly lower blood glucose levels than P14 pups. The average blood glucose concentration at P1 is 3 mM, whereas blood glucose at P14 averages 6.2 mM (P<2.5×10-24) (FIG. 1d). Notably, the average blood glucose level in P1 pups is higher than the glucose concentration that causes insulin secretion in vitro in P1 islets. If the in vitro observation that immature β-cells secrete insulin at low glucose levels (FIG. 1a) holds true in vivo, one should see higher insulin in the blood of neonates. Consistent with this prediction, the P1 pups had nearly two-fold higher levels of insulin in their blood than P14 animals (FIG. 1e), although we note that there is a high variability of blood insulin in non-fasted animals. The present inventors also examined insulin granules in β-cells at each stage using electron microscopy (FIG. 1f,g). P1 β-cells contained approximately twofold fewer insulin granules compared with P10 β-cells, suggesting a mechanistic difference in insulin secretion.

To find molecular markers whose expression pattern correlates with β-cell maturation, the present inventors sorted β cells expressing Pdx1-EGFP from P1 or P10 animals by fluorescence-activated cell sorting (FACS) and compared their global gene expression patterns using transcriptional arrays. The Pdx1-EGFP strain was used instead of the insulin-EGFP strain as the latter animals were slightly diabetic. The present inventors also analyzed β cells from E18.5 embryos and adult mice (FIG. 1h) to further reduce the number of genes whose transcriptional differences are not related to GSIS maturation. Notably, the gene expression profiles of functionally mature and immature p cells tested this way are very similar (FIG. 1i).

The present inventors investigated known β-cell genes whose expression levels could explain the functional difference between mature and immature β-cells. In particular, the present inventors examined expression levels of β-cell transcription factors (Pdx1, Nkx2.2, Nkx6.1, NeuroD1, Foxa1, Foxa2, MafA, MafB and Hnf4a), key proteins involved in glucose sensing and insulin secretion (Glucokinase, Glut2, Cav6.1, Kir6.1, Sur1, Pcsk1 and Pcsk2), the β-cell-selective gap junction Connexin36, and Insulin1 and Insulin2. Tissue-specific glucose transporters (Glut 1, 3 and 4) and hexokinases (Hexokinase 1, 2 and 3) (FIG. 1j) were also examined. Remarkably, the RNA expression levels of most of these genes did not change significantly between immature and mature cells (or change expression by less than twofold, making them unsuitable for on/off detection of mature β-cells), except for expression of the transcription factor MafB, which is expressed at 2.5-fold higher levels in immature β-cells, consistent with previous reports (Artner et al. Diabetes 59, 2530-2539 (2010)).

We next examined all genes for which expression changes by more than twofold between immature and mature cells. We excluded genes for which a significant change in expression also occurred between the younger mice, E18.5 and P1, and the older mice, P10 and adult, thereby focusing on genes that change expression specifically within the time window of β-cell maturation (groups i and ii in FIG. 1h). We found 71 genes (81 probes) that were upregulated and 66 genes (72 probes) that were downregulated during β-cell maturation. Of the former group, 36 genes were acinar-related genes (Table 1), which is best explained by the rapid expansion of exocrine tissue at this stage, thereby increasing the probability of a small acinar cell contamination during FACS sorting and resulting in the misleading indication that acinar genes are upregulated. We chose 16 genes (Table 1) for which β-cell expression had previously been reported and analyzed their protein expression levels using western blot analysis and immunohistochemistry.

Remarkably, the levels of UCN3 mRNA increased more than sevenfold between immature and mature β-cells, and nearly tenfold between E18.5 and adult (FIG. 1k and Table 1). Immunofluorescence staining showed that UCN3 was highly expressed in all adult β-cells, but was undetectable in islets from E18.5 embryos (FIG. 1l,m). As with insulin, the signal intensity of UCN3 protein varies from cell to cell in the adult islet. This variation does not correlate with the variation in insulin intensity as cells that show high staining intensity for insulin show both high and low staining intensities for UCN3, and vice versa. No co-localization of UCN3 with glucagon, somatostatin or pancreatic polypeptide (PPY) was observed, indicating β-cell-specific expression of the gene (FIG. 3).

UCN3 is a secreted protein expressed in regions of the brain and in the pancreas, and was reported to be exclusively expressed by β-cells but not by other endocrine cells in the islet (Li et al. Endocrinology 144, 3216-3224 (2003)). Secretion of UCN3 from β-cells is induced by high glucose in adult mice, and the gene has a positive effect on GSIS at high glucose concentrations (Li et al. 2003; Li et al. PNAS USA 104, 4206-4211 (2007)).

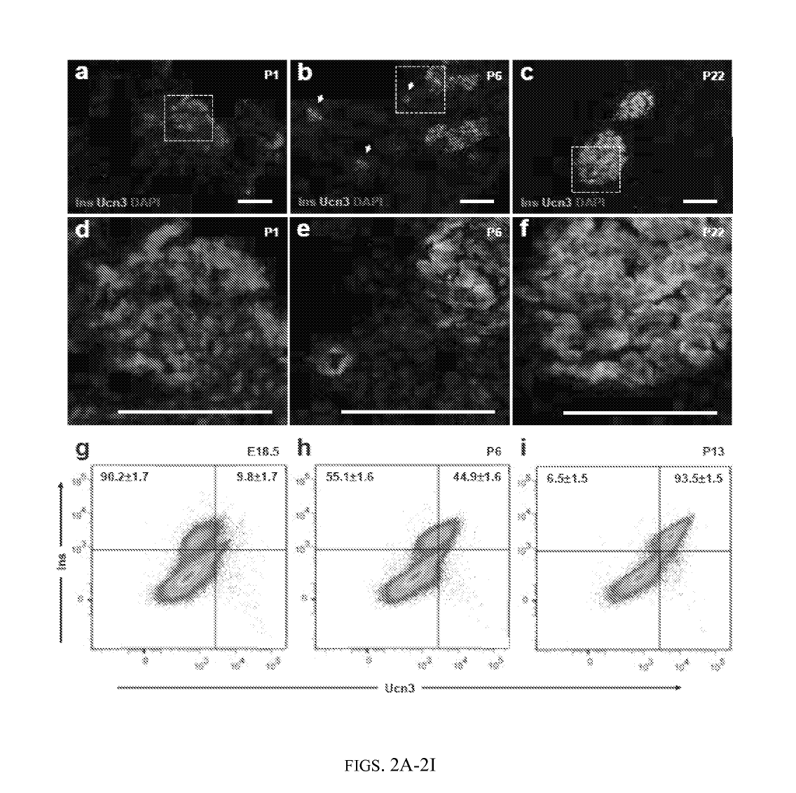

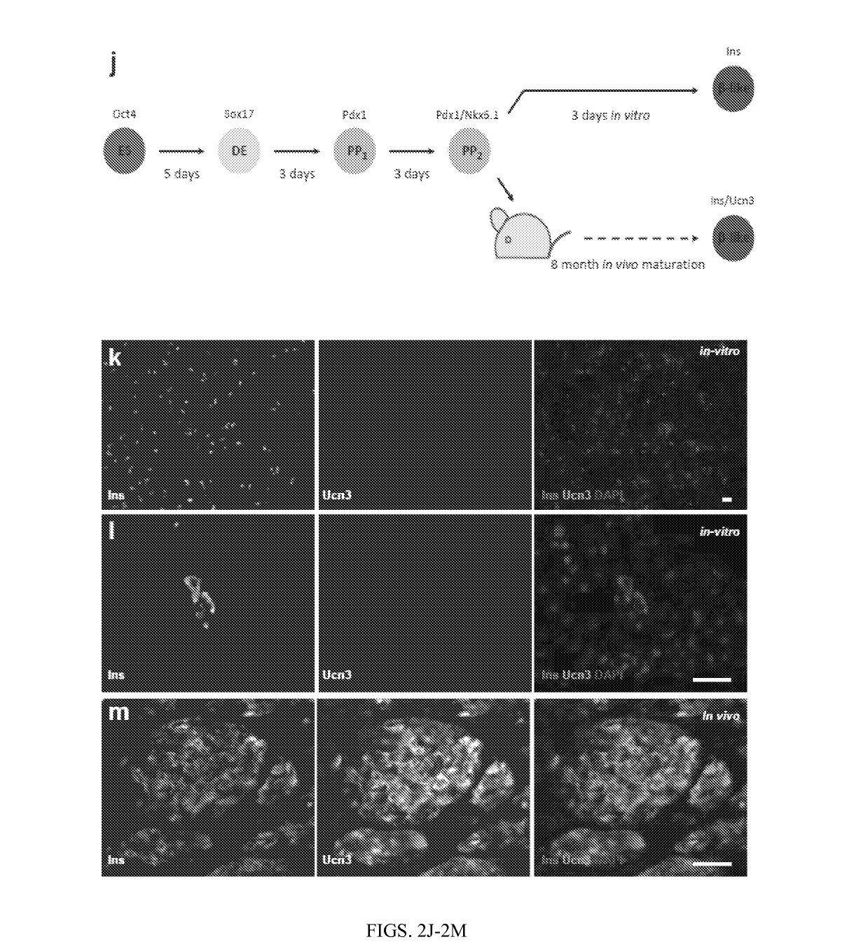

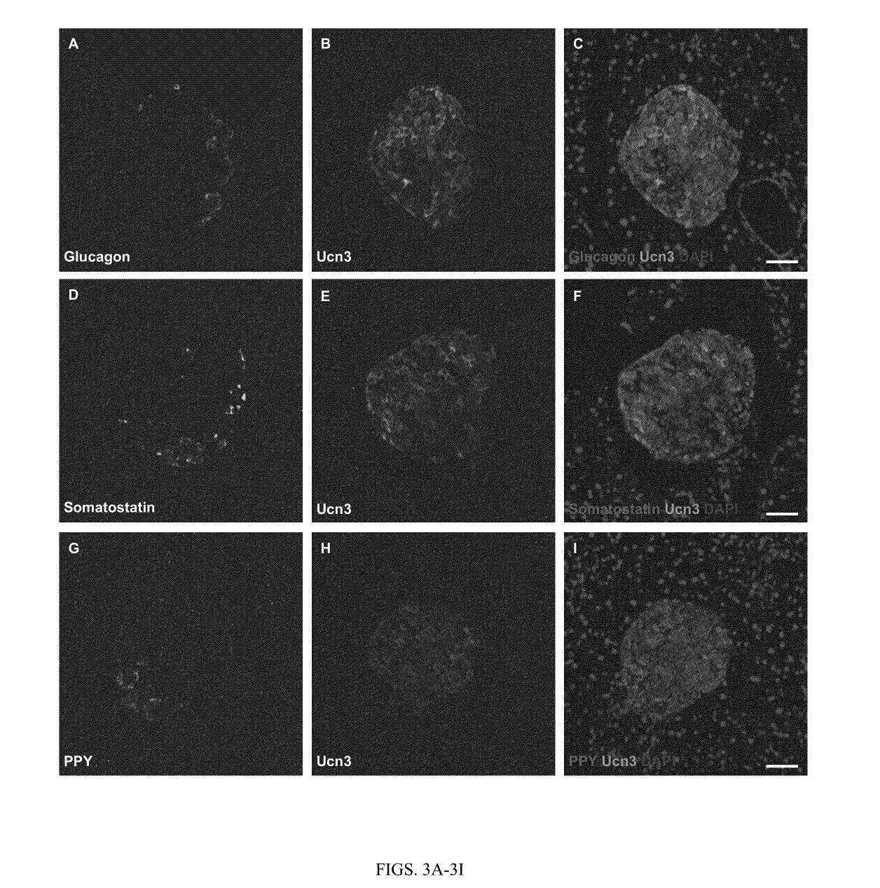

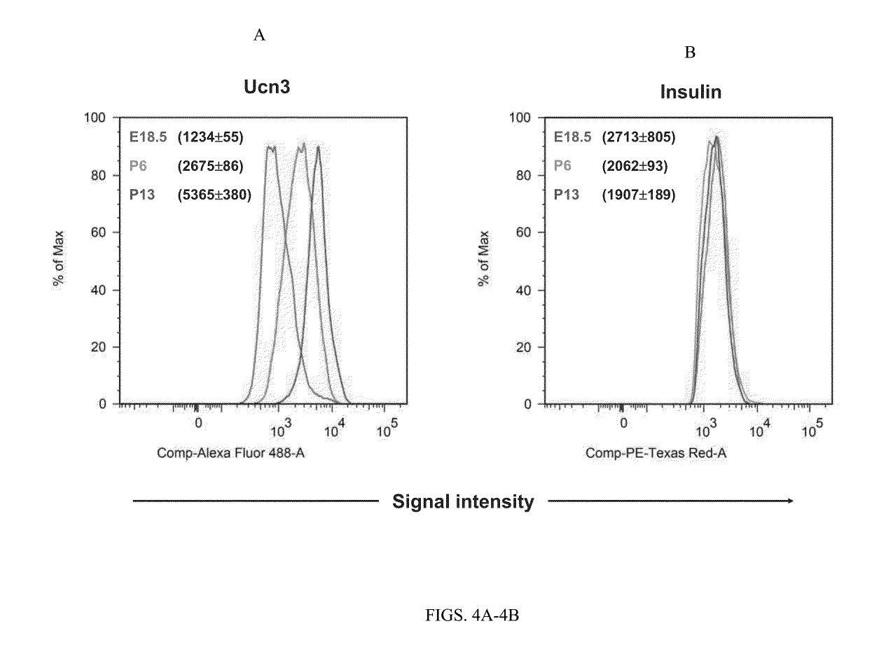

We next examined the patterns of UCN3 expression at additional time points during the period of β-cell maturation. UCN3 protein was not detected in any islets of P1 pups (FIG. 2a-f). At P6, UCN3 expression is found primarily in large islets, not in small β-cell aggregates. By P22, UCN3 protein is strongly detected in all β-cells. Intracellular FACS analysis with antibodies against UCN3 and insulin was done to quantify the percentage and levels of UCN3 expression in β-cells. At 518.5, 90.2±1.7% of β-cells express insulin alone, and 9.8±1.7% also stain weakly for UCN3 (FIG. 2g). The low expression level of UCN3 in the small population detected by FACS at this age is probably too low to be detected by conventional immunofluorescence on tissue sections. At P6, 55.1±1.6% of β-cells are either negative for UCN3 or express low levels of the protein, whereas 44.9±1.6% of β-cells express high levels of both UCN3 and insulin (FIG. 2h). By P13, just at the end of the maturation window, 93.5±1.5% of the β-cells express high levels of UCN3 and only 6.5±1.5% express insulin alone (FIG. 2i). The increase in UCN3 in β-cells during maturation is gradual, as can be seen by the shift in the mean UCN3 signal intensity (FIG. 4a). The signal intensity of insulin is unchanged, indicating that expression of insulin protein remains constant throughout this time period (FIG. 4b). This mixed pattern of UCN3 expression may explain why a marginally mature phenotype was observed between P2 and P9.

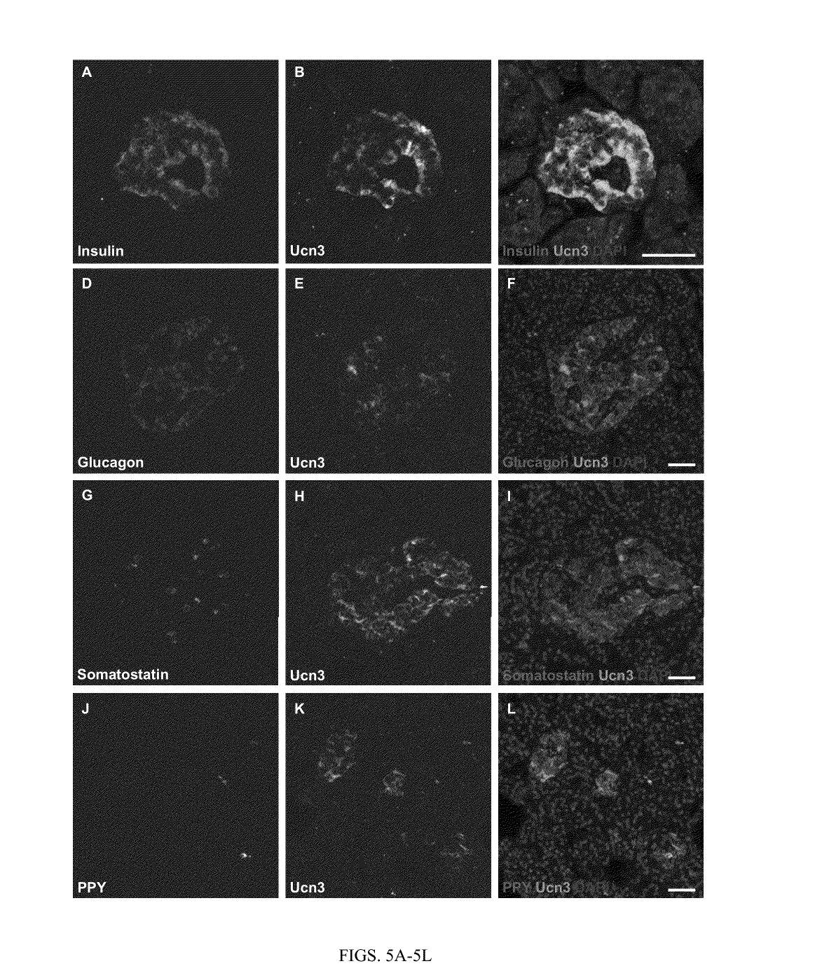

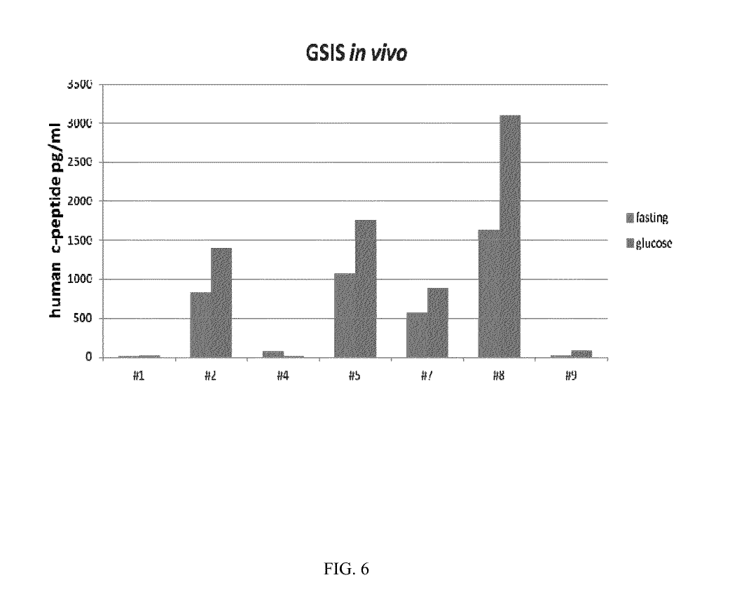

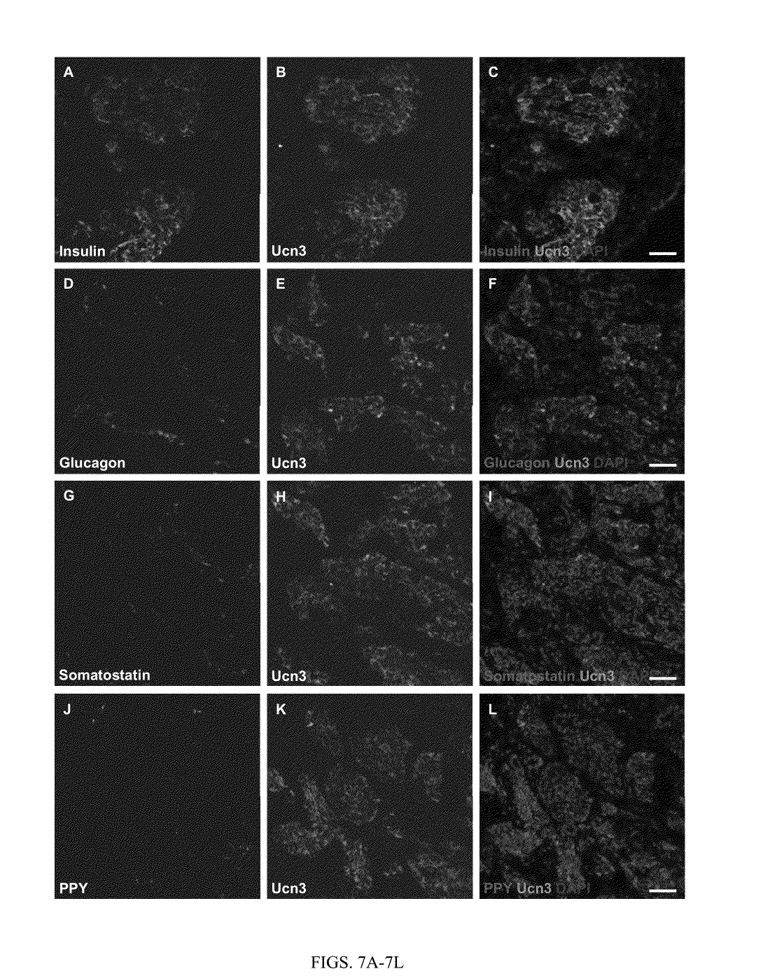

During the course of work described herein, the present inventors demonstrated that UCN3 can serve as a marker for functionally mature β-cells derived from stem cells (e.g., hPSCs). Immunoassaying with antibodies against UCN3 on pancreatic sections obtained from an adult human donor revealed that the gene is expressed by all insulin-positive β-cells and not expressed in glucagon-expressing alpha cells. A small fraction of somatostatin- and PPY-expressing cells also expressed UCN3 (FIG. 5). To investigate whether UCN3 is induced during the maturation of human embryonic stem cell (hESC)-derived β-cells after transplantation, hESCs were differentiated using a four-step protocol to Pdx1+ and NKX6.1+ pancreatic progenitors. These cells were then differentiated in vitro for 3 more days to become insulin-positive β-like cells (FIG. 2j and Materials and Methods). Separately, stage 4 clusters of Pdx+Nkx6.1+ pancreatic progenitors, containing a few insulin-positive p-like cells, were transplanted to the kidney capsule of severe combined immunodeficient (SCID)-beige mice, where they differentiate further and mature in vivo (FIG. 2j). A glucose tolerance test done on transplanted animals showed fasting human C-peptide levels above background 12 weeks after transplantation (FIG. 6). Despite high variation in fasting human C-peptide between the transplanted mice, all but one animal (6/7) showed an increase in blood human C-peptide of 1.7-fold to 7.6-fold (average 2.8-±0.9-fold), demonstrating that the transplanted hESC-derived cells matured to glucose-responsive β-cells. Immunostaining showed that although the in vitro-differentiated p-like cells expressed insulin, they were negative for UCN3 staining (FIG. 2k,l). Conversely, the in vivo-matured cells stained positive for both insulin and UCN3 proteins (FIG. 2m). This expression of human UCN3 in the transplant was exclusive to the β-cells; the UCN3 protein was not detected in any glucagon-, somatostatin- or PPY-expressing cells (FIG. 7).

In summary, the studies described above provide an operational definition for mature β-cells based on changing glucose thresholds for GSIS response during development. The studies described above also demonstrate that UCN3 is a molecular marker that distinguishes mature and immature β-cells. In this regard, UCN3 is induced in hESC-derived β-cells after maturation in vivo. On the basis of the work described herein, the difference in GSIS and the expression of UCN3 can be used in methods of identifying agents that induce functional β-cell maturation in vitro.

TABLE 1

| | | | | | | |
|---|---|---|---|---|---|---|
| | | | | | | Fold |
| PROBE_ID | Gene symbol | E18.5 | P1 | P10 | Adult | change |

Up regulated genes

| | | | | | | |
|---|---|---|---|---|---|---|
| ILMN_2673260 | Ctrl* | 49 ± 8 | 69 ± 30 | 2269 ± 2057 | 3110 ± 1910 | 59.1 |
| ILMN_2881083 | Try10* | 65 ± 29 | 300 ± 312 | 4572 ± 3844 | 7237 ± 4442 | 39.7 |
| ILMN_2716989 | Prss2* | 46 ± 6 | 81 ± 54 | 1663 ± 1192 | 2392 ± 1334 | 39.4 |
| ILMN_3160208 | Cpb1* | 48 ± 17 | 83 ± 51 | 1627 ± 1322 | 2169 ± 1747 | 34.7 |
| ILMN_2693403 | Ela1* | 88 ± 26 | 102 ± 33 | 1513 ± 758 | 5071 ± 2642 | 34.0 |
| ILMN_2666677 | Cel* | 60 ± 23 | 95 ± 48 | 2100 ± 1965 | 1871 ± 1213 | 33.4 |
| ILMN_2493756 | Try4* | 49 ± 14 | 156 ± 141 | 1985 ± 1574 | 3706 ± 2554 | 33.1 |
| ILMN_2728429 | 1810010M01Rik* | 53 ± 14 | 90 ± 52 | 1051 ± 632 | 3239 ± 2226 | 31.5 |
| ILMN_2592415 | Reg1* | 106 ± 71 | 202 ± 141 | 4954 ± 3212 | 3272 ± 2018 | 30.1 |
| ILMN_2671137 | Ela3b* | 57 ± 27 | 125 ± 94 | 1752 ± 1573 | 2280 ± 1351 | 26.7 |
| ILMN_1220763 | Rnase1* | 69 ± 38 | 128 ± 90 | 1891 ± 1290 | 3065 ± 2175 | 26.7 |
| ILMN_2670847 | Cpa1* | 89 ± 38 | 107 ± 52 | 1718 ± 1287 | 2728 ± 1855 | 24.6 |
| ILMN_3104915 | 1810049H19Rik* | 47 ± 12 | 141 ± 122 | 1493 ± 1201 | 1953 ± 1200 | 23.3 |
| ILMN_2919377 | Ctrb1* | 106 ± 59 | 225 ± 203 | 3360 ± 2265 | 2670 ± 2042 | 21.2 |
| ILMN_2933478 | Amy2-2* | 47 ± 9 | 83 ± 43 | 428 ± 228 | 2166 ± 1554 | 20.2 |
| ILMN_1246265 | Clps* | 233 ± 182 | 467 ± 419 | 6237 ± 3526 | 6551 ± 4551 | 18.5 |
| ILMN_2963762 | Try10l* | 50 ± 15 | 163 ± 155 | 1272 ± 1049 | 1887 ± 1138 | 18.5 |
| ILMN_2874291 | Amy2* | 81 ± 45 | 205 ± 186 | 1080 ± 588 | 4393 ± 2661 | 18.3 |
| ILMN_1216509 | Ctrc* | 40 ± 4 | 43 ± 8 | 274 ± 196 | 1084 ± 920 | 17.9 |
| ILMN_2990661 | Pnliprp2* | 38 ± 5 | 47 ± 13 | 502 ± 355 | 697 ± 568 | 17.3 |
| ILMN_2722659 | Clps* | 86 ± 43 | 149 ± 113 | 1829 ± 1128 | 1668 ± 1335 | 16.7 |
| ILMN_2674620 | Ela2* | 253 ± 286 | 627 ± 576 | 5169 ± 3159 | 10910 ± 5518 | 16.5 |
| ILMN_1232533 | Sycn* | 46 ± 12 | 72 ± 34 | 462 ± 292 | 1374 ± 1022 | 16.5 |
| ILMN_2829699 | EG436523* | 51 ± 24 | 167 ± 160 | 1127 ± 957 | 1320 ± 996 | 14.1 |
| ILMN_2708477 | Spink3* | 52 ± 13 | 98 ± 76 | 993 ± 641 | 529 ± 383 | 13.3 |
| ILMN_2760199 | Klk6 | 57 ± 13 | 74 ± 23 | 564 ± 384 | 829 ± 609 | 12.3 |
| ILMN_2904435 | Gp2* | 37 ± 1 | 41 ± 7 | 181 ± 80 | 622 ± 493 | 11.2 |
| ILMN_1225909 | Pnliprp1* | 451 ± 380 | 774 ± 664 | 8347 ± 1724 | 4806 ± 2403 | 9.3 |
| ILMN_2844820 | Angptl7 | 68 ± 4 | 105 ± 16 | 383 ± 125 | 1043 ± 151 | 8.8 |
| ILMN_1216566 | LOC232680* | 61 ± 9 | 64 ± 13 | 452 ± 198 | 447 ± 349 | 8.2 |
| ILMN_2731191 | Klk5* | 46 ± 4 | 49 ± 10 | 238 ± 157 | 343 ± 269 | 7.5 |
| ILMN_2684115 | 2210010C04Rik* | 37 ± 2 | 48 ± 13 | 201 ± 147 | 297 ± 184 | 7.4 |
| ILMN_1238143 | Ucn3 | 127 ± 20 | 154 ± 13 | 923 ± 22 | 1234 ± 285 | 7.2 |
| ILMN_2690014 | Syt4 | 80 ± 4 | 62 ± 5 | 252 ± 22 | 734 ± 107 | 6.9 |
| ILMN_2692167 | Pnliprp2* | 40 ± 2 | 43 ± 7 | 176 ± 130 | 264 ± 205 | 6.7 |
| ILMN_1226556 | 2310032F03Rik* | 44 ± 3 | 47 ± 4 | 172 ± 15 | 435 ± 207 | 6.6 |
| ILMN_2860932 | Zbtb2* | 91 ± 13 | 76 ± 9 | 348 ± 211 | 629 ± 600 | 6.6 |
| ILMN_1259215 | Serpina10 | 136 ± 10 | 97 ± 25 | 275 ± 18 | 1303 ± 13 | 6.6 |
| ILMN_1252131 | Klk1b27 | 44 ± 5 | 52 ± 13 | 193 ± 151 | 270 ± 216 | 6.1 |
| ILMN_1228211 | Tff2 | 95 ± 3 | 81 ± 17 | 409 ± 215 | 446 ± 334 | 6.0 |
| ILMN_1238736 | Klk1b4 | 50 ± 3 | 58 ± 15 | 211 ± 141 | 308 ± 225 | 5.9 |
| ILMN_1231724 | Resp18 | 577 ± 54 | 684 ± 113 | 2693 ± 221 | 4401 ± 528 | 5.6 |
| ILMN_2850077 | Adh1 | 159 ± 21 | 195 ± 18 | 654 ± 25 | 1232 ± 221 | 5.1 |
| ILMN_2824971 | Gpr158* | 149 ± 7 | 163 ± 25 | 617 ± 88 | 900 ± 134 | 5.0 |
| ILMN_2592718 | Cuzd1* | 39 ± 4 | 39 ± 2 | 104 ± 58 | 235 ± 167 | 4.9 |
| ILMN_1258501 | Adh1 | 141 ± 23 | 161 ± 7 | 555 ± 19 | 999 ± 242 | 4.8 |
| ILMN_2871660 | Car15 | 52 ± 4 | 55 ± 1 | 364 ± 35 | 120 ± 45 | 4.7 |
| ILMN_2968692 | Cpa2 | 41 ± 5 | 44 ± 3 | 164 ± 80 | 152 ± 94 | 4.4 |
| ILMN_2960700 | Prf1 | 277 ± 43 | 249 ± 43 | 821 ± 302 | 1281 ± 918 | 4.2 |
| ILMN_2804685 | Defb1 | 69 ± 6 | 133 ± 42 | 319 ± 19 | 536 ± 126 | 4.2 |
| ILMN_2596522 | Mt1* | 958 ± 521 | 1348 ± 109 | 2719 ± 1045 | 7849 ± 4383 | 4.1 |
| ILMN_2822825 | Fbxo2 | 90 ± 1 | 111 ± 24 | 314 ± 50 | 401 ± 27 | 3.8 |
| ILMN_2695819 | Ddit4l | 49 ± 4 | 50 ± 4 | 168 ± 51 | 149 ± 46 | 3.6 |
| ILMN_2728038 | Arhgap24 | 139 ± 16 | 128 ± 28 | 348 ± 33 | 595 ± 215 | 3.4 |
| ILMN_2648742 | Abcb4 | 78 ± 20 | 90 ± 19 | 266 ± 14 | 362 ± 46 | 3.4 |
| ILMN_2839027 | Tceal6 | 95 ± 9 | 102 ± 21 | 238 ± 10 | 447 ± 41 | 3.4 |
| ILMN_2994299 | Hgfac | 182 ± 43 | 243 ± 18 | 547 ± 46 | 965 ± 91 | 3.3 |
| ILMN_2628647 | Ddc | 560 ± 97 | 553 ± 76 | 1408 ± 133 | 2434 ± 256 | 3.3 |
| ILMN_3064283 | Pde4dip | 115 ± 3 | 93 ± 17 | 245 ± 7 | 433 ± 45 | 3.3 |
| ILMN_2681232 | D12Ertd647e | 216 ± 19 | 218 ± 20 | 492 ± 31 | 931 ± 203 | 3.2 |
| ILMN_1254335 | Rgpr | 50 ± 3 | 47 ± 4 | 112 ± 13 | 194 ± 27 | 3.2 |
| ILMN_3108770 | Fbxl10 | 117 ± 18 | 116 ± 19 | 309 ± 1 | 489 ± 51 | 3.2 |
| ILMN_1250689 | Rgs9 | 133 ± 27 | 107 ± 14 | 361 ± 22 | 456 ± 49 | 3.1 |
| ILMN_2624854 | Gstm2 | 146 ± 23 | 140 ± 28 | 316 ± 36 | 603 ± 61 | 3.1 |
| ILMN_1251449 | Gstm2 | 144 ± 21 | 126 ± 11 | 303 ± 16 | 578 ± 64 | 3.1 |
| ILMN_2601471 | Ccnd1 | 177 ± 64 | 196 ± 13 | 473 ± 92 | 763 ± 165 | 3.0 |
| ILMN_2959272 | Rnu6 | 669 ± 62 | 511 ± 128 | 1627 ± 739 | 1383 ± 630 | 3.0 |
| ILMN_3162403 | St6galnac3 | 70 ± 4 | 79 ± 13 | 175 ± 3 | 280 ± 14 | 3.0 |
| ILMN_2647234 | Dio1 | 62 ± 9 | 65 ± 7 | 135 ± 13 | 248 ± 98 | 2.9 |
| ILMN_3125606 | D12Ertd647e | 215 ± 22 | 214 ± 2 | 449 ± 29 | 800 ± 78 | 2.8 |
| ILMN_2856567 | Ppy | 1597 ± 704 | 2056 ± 957 | 5681 ± 1998 | 4387 ± 3417 | 2.8 |
| ILMN_2966162 | Tmem56 | 88 ± 8 | 74 ± 20 | 188 ± 17 | 266 ± 91 | 2.8 |
| ILMN_1217118 | Enpp5 | 280 ± 46 | 281 ± 19 | 587 ± 2 | 1071 ± 343 | 2.7 |
| ILMN_1221503 | Ccnd1 | 206 ± 81 | 207 ± 21 | 466 ± 97 | 788 ± 134 | 2.7 |
| ILMN_2862470 | Gstm2 | 81 ± 11 | 77 ± 12 | 163 ± 0 | 270 ± 21 | 2.6 |
| ILMN_2646640 | 1700019D03Rik | 84 ± 7 | 91 ± 18 | 185 ± 2 | 278 ± 53 | 2.6 |
| ILMN_2615096 | Dpp4 | 119 ± 20 | 106 ± 17 | 312 ± 0 | 306 ± 19 | 2.5 |
| ILMN_2652757 | Elovl5 | 501 ± 20 | 510 ± 89 | 1153 ± 120 | 1247 ± 103 | 2.4 |
| ILMN_2722996 | Ptpns1 | 407 ± 77 | 424 ± 89 | 883 ± 13 | 1240 ± 203 | 2.4 |
| ILMN_2731769 | Plekhb2 | 149 ± 20 | 161 ± 24 | 341 ± 35 | 365 ± 60 | 2.2 |
| ILMN_2652414 | Ncald | 146 ± 13 | 141 ± 23 | 312 ± 12 | 337 ± 31 | 2.2 |

Down regulated genes

| | | | | | | |
|---|---|---|---|---|---|---|
| ILMN_1244618 | Dlk1 | 2028 ± 195 | 2051 ± 315 | 126 ± 18 | 39 ± 1 | −23.3 |
| ILMN_2946520 | Npy | 3464 ± 545 | 2001 ± 551 | 235 ± 25 | 77 ± 38 | −17.8 |
| ILMN_2643658 | Ghrl | 1199 ± 617 | 643 ± 205 | 77 ± 27 | 40 ± 4 | −17.1 |
| ILMN_2755578 | Nnat | 1677 ± 588 | 1286 ± 499 | 257 ± 79 | 69 ± 17 | −8.8 |
| ILMN_2649773 | Slc38a5 | 7037 ± 586 | 5761 ± 660 | 1181 ± 68 | 421 ± 122 | −8.0 |
| ILMN_1232456 | Nnat | 593 ± 146 | 448 ± 102 | 98 ± 28 | 42 ± 2 | −7.1 |
| ILMN_2598022 | Ghrl | 337 ± 147 | 173 ± 38 | 50 ± 2 | 43 ± 2 | −6.9 |
| ILMN_2518412 | Grb10 | 1459 ± 160 | 933 ± 133 | 246 ± 11 | 155 ± 50 | −6.2 |
| ILMN_2635700 | Lgi2 | 823 ± 136 | 833 ± 113 | 131 ± 10 | 166 ± 63 | −5.8 |
| ILMN_1251414 | Nxf | 525 ± 159 | 510 ± 141 | 89 ± 27 | 94 ± 48 | −5.7 |
| ILMN_2643049 | Chst8 | 419 ± 65 | 611 ± 109 | 113 ± 16 | 64 ± 7 | −5.7 |
| ILMN_2842601 | Gp9 | 356 ± 64 | 240 ± 49 | 62 ± 15 | 48 ± 3 | −5.3 |
| ILMN_1215713 | Egr4 | 514 ± 250 | 434 ± 157 | 130 ± 51 | 46 ± 4 | −5.3 |
| ILMN_2597769 | Igf2 | 1412 ± 843 | 852 ± 433 | 302 ± 106 | 232 ± 92 | −4.9 |
| ILMN_2906728 | H19 | 239 ± 186 | 122 ± 107 | 54 ± 18 | 41 ± 2 | −4.8 |
| ILMN_2629519 | Cryba2 | 4385 ± 749 | 4145 ± 949 | 1153 ± 153 | 624 ± 50 | −4.8 |
| ILMN_2623983 | Egr2 | 247 ± 110 | 186 ± 96 | 63 ± 10 | 45 ± 5 | −4.6 |
| ILMN_2597827 | Arc | 321 ± 147 | 288 ± 91 | 90 ± 29 | 46 ± 8 | −4.6 |
| ILMN_2619408 | Atf3 | 198 ± 110 | 115 ± 55 | 47 ± 6 | 43 ± 4 | −4.4 |
| ILMN_2734712 | Ptpla | 551 ± 81 | 372 ± 48 | 139 ± 5 | 87 ± 13 | −4.3 |
| ILMN_2708203 | Cdkn1c | 262 ± 67 | 203 ± 34 | 67 ± 2 | 55 ± 3 | −4.3 |
| ILMN_1250438 | Mlp | 233 ± 41 | 190 ± 64 | 59 ± 7 | 47 ± 1 | −4.1 |
| ILMN_2592834 | Sct | 274 ± 35 | 177 ± 23 | 68 ± 3 | 49 ± 3 | −4.0 |
| ILMN_1218913 | Igf2bp3 | 307 ± 30 | 254 ± 45 | 94 ± 15 | 39 ± 1 | −4.0 |
| ILMN_2834379 | Tgfbi | 175 ± 30 | 155 ± 8 | 47 ± 5 | 41 ± 4 | −3.9 |
| ILMN_2745551 | Olfml2b | 228 ± 23 | 133 ± 38 | 45 ± 6 | 48 ± 5 | −3.8 |
| ILMN_2687661 | Mfng | 385 ± 25 | 427 ± 76 | 157 ± 29 | 41 ± 9 | −3.7 |
| ILMN_2945030 | Col9a2 | 258 ± 18 | 218 ± 30 | 82 ± 4 | 52 ± 2 | −3.6 |
| ILMN_2981542 | Mfap2 | 189 ± 53 | 171 ± 50 | 59 ± 4 | 51 ± 8 | −3.6 |
| ILMN_2771237 | Lbp | 161 ± 119 | 251 ± 167 | 70 ± 4 | 74 ± 45 | −3.6 |
| ILMN_1226175 | Igf2bp3 | 255 ± 16 | 187 ± 25 | 85 ± 4 | 39 ± 4 | −3.6 |
| ILMN_2718330 | Cish | 1136 ± 211 | 1235 ± 112 | 421 ± 78 | 226 ± 92 | −3.6 |
| ILMN_2747543 | Actn3 | 192 ± 23 | 217 ± 37 | 66 ± 4 | 52 ± 4 | −3.5 |
| ILMN_1221750 | Lmyc1 | 193 ± 18 | 216 ± 22 | 73 ± 9 | 39 ± 1 | −3.5 |
| ILMN_2900653 | Gadd45b | 396 ± 125 | 306 ± 38 | 105 ± 50 | 87 ± 18 | −3.4 |
| ILMN_2636403 | Axud1 | 248 ± 155 | 169 ± 23 | 34 ± 11 | 75 ± 19 | −3.4 |
| ILMN_1244169 | Sftpd | 244 ± 32 | 258 ± 55 | 87 ± 6 | 66 ± 8 | −3.4 |
| ILMN_1222084 | Rem2 | 564 ± 60 | 634 ± 197 | 229 ± 47 | 112 ± 47 | −3.2 |
| ILMN_1225764 | 1700018O18Rik | 133 ± 18 | 150 ± 11 | 47 ± 4 | 43 ± 5 | −3.2 |
| ILMN_2828112 | Igfbpl1 | 134 ± 12 | 106 ± 8 | 38 ± 2 | 38 ± 2 | −3.2 |
| ILMN_2776034 | Gal | 202 ± 18 | 185 ± 20 | 48 ± 5 | 75 ± 19 | −3.2 |
| ILMN_2445324 | Zfpm1 | 426 ± 58 | 489 ± 80 | 205 ± 3 | 105 ± 22 | −3.1 |
| ILMN_2941790 | Cldn6 | 433 ± 86 | 344 ± 39 | 148 ± 9 | 123 ± 45 | −3.1 |
| ILMN_2736478 | Doc2b | 187 ± 25 | 134 ± 52 | 63 ± 3 | 47 ± 2 | −3.1 |
| ILMN_2930602 | Doc2b | 176 ± 39 | 114 ± 54 | 57 ± 7 | 44 ± 4 | −3.0 |
| ILMN_1246201 | Cacna1h | 218 ± 44 | 141 ± 54 | 62 ± 3 | 68 ± 21 | −3.0 |
| ILMN_2514292 | Zyx | 355 ± 86 | 264 ± 64 | 108 ± 16 | 112 ± 20 | −3.0 |
| ILMN_2678714 | Idb4 | 195 ± 28 | 195 ± 30 | 84 ± 14 | 43 ± 1 | −3.0 |
| ILMN_2638324 | Gnas | 105 ± 34 | 121 ± 13 | 45 ± 4 | 40 ± 6 | −2.9 |
| ILMN_2700608 | Stx1a | 310 ± 45 | 341 ± 16 | 138 ± 12 | 93 ± 15 | −2.9 |
| ILMN_2718217 | 2310057H16Rik | 248 ± 58 | 195 ± 36 | 91 ± 3 | 80 ± 5 | −2.9 |
| ILMN_2715546 | Gpx3 | 3096 ± 199 | 3125 ± 456 | 1018 ± 207 | 1034 ± 229 | −2.8 |
| ILMN_2731407 | Gdf3 | 131 ± 23 | 108 ± 15 | 44 ± 1 | 48 ± 6 | −2.8 |
| ILMN_1240857 | Cox7a1 | 149 ± 26 | 128 ± 28 | 58 ± 3 | 46 ± 5 | −2.8 |
| ILMN_2714031 | 1300002F13Rik | 716 ± 184 | 576 ± 164 | 278 ± 3 | 246 ± 42 | −2.8 |
| ILMN_1256702 | S100a10 | 574 ± 73 | 455 ± 94 | 157 ± 36 | 200 ± 72 | −2.8 |
| ILMN_2756704 | 9130213B05Rik | 195 ± 32 | 161 ± 27 | 78 ± 1 | 61 ± 4 | −2.8 |
| ILMN_2728379 | Ivd | 852 ± 99 | 1010 ± 106 | 368 ± 11 | 325 ± 51 | −2.8 |
| ILMN_2604029 | Klf2 | 252 ± 98 | 187 ± 63 | 89 ± 42 | 66 ± 16 | −2.7 |
| ILMN_2632206 | Gnas | 1494 ± 455 | 1575 ± 250 | 708 ± 41 | 609 ± 135 | −2.6 |
| ILMN_2731949 | Copeb | 273 ± 86 | 238 ± 81 | 108 ± 33 | 91 ± 33 | −2.6 |
| ILMN_2430220 | Tmem2 | 174 ± 34 | 164 ± 16 | 77 ± 5 | 65 ± 6 | −2.5 |
| ILMN_1248740 | Sema3f | 138 ± 31 | 123 ± 25 | 60 ± 7 | 49 ± 6 | −2.5 |
| ILMN_2766651 | Mafb | 115 ± 11 | 104 ± 33 | 47 ± 3 | 41 ± 2 | −2.5 |
| ILMN_3008110 | Actn3 | 136 ± 15 | 141 ± 22 | 64 ± 2 | 57 ± 5 | −2.4 |
| ILMN_2735184 | Col18a1 | 259 ± 79 | 210 ± 61 | 92 ± 56 | 86 ± 6 | −2.4 |
| ILMN_2996683 | Pvrl2 | 376 ± 21 | 320 ± 43 | 159 ± 16 | 134 ± 34 | −2.3 |
| ILMN_2697361 | B930096L08Rik | 121 ± 16 | 120 ± 21 | 60 ± 6 | 47 ± 5 | −2.3 |
| ILMN_2745614 | 1810015C04Rik | 278 ± 37 | 248 ± 94 | 122 ± 4 | 122 ± 33 | −2.3 |
| ILMN_3133448 | Mfge8 | 412 ± 138 | 409 ± 52 | 165 ± 87 | 195 ± 77 | −2.1 |
| ILMN_2646625 | Jun | 456 ± 106 | 460 ± 158 | 220 ± 74 | 196 ± 90 | −2.1 |
| ILMN_2696299 | D5Ertd579e | 509 ± 27 | 523 ± 128 | 245 ± 50 | 245 ± 51 | −2.0 |

1. A method of determining the functional maturity of a β-cell or a population of β-cells, comprising:
(a) obtaining a β-cell or a population of β-cells;
(b) assaying the β-cell or population of β-cells for the presence or absence of one or more of: a GSIS response at low glucose concentrations, a large fold change in the GSIS response between low and high glucose concentrations, urocortin 3 (UCN3) protein, and elevated levels of UCN3 messenger ribonucleic acid (mRNA); and
(c) determining the functional maturity of the β-cell or population of β-cells, wherein the β-cell or population of β-cells is:
(i) functionally immature if the β-cell or β-cells in the population exhibit one or more of the presence of a GSIS response at low glucose concentrations, the absence of a large fold change in the GSIS response between low and high glucose concentrations, the absence of UCN3 protein, or the absence of elevated levels of UCN3 mRNA; or
(ii) functionally mature if the β-cell or β-cells in the population exhibit one or more of the absence of a GSIS response at low glucose concentrations, the presence of a large fold change in the GSIS response between low and high glucose concentrations, the presence of UCN3 protein, or the presence of elevated levels of UCN3 mRNA.

2. The method of claim 1 wherein the β-cell or population of β-cells are obtained from an in vitro source.

3. The method of claim 2 wherein the in vitro source is a culture of differentiating stem cells.

4. The method of claim 3 wherein the stem cells are selected from the group consisting of human embryonic stem cells (hESCs), induced pluripotent stem cells (iPSCs), and combinations thereof.

5-6. (canceled)

7. The method of claim 1 wherein the fβ-cell is obtained from an in vivo source selected from the group consisting of (i) an individual who has received an administration of β-cells (ii) an individual suffering from a disorder associated with immature β-cells (iii) an individual suspected of being in need of functionally mature β-cells and (iv) a tissue or organ obtained from a donor individual.

8-13. (canceled)

14. The method of claim 1 wherein the absence of the GSIS response at low glucose concentrations is a lack of insulin secretion in response to the low glucose concentrations.

15. The method of claim 1 wherein the low glucose concentration is less than or equal to about 5 mM.

16. (canceled)

17. The method of claim 1 wherein the high glucose concentration is greater than or equal to about 10 mM.

18. (canceled)

19. The method of claim 1 wherein the large fold change in the GSIS response between the low and high glucose concentrations is at least about 2.5 fold.

20. The method of claim 1 wherein the large fold change in the GSIS response between the low and high glucose concentrations is greater than or equal to about 50 fold.

21. The method of claim 1 wherein assaying the β-cell or population of β-cells for the presence or absence of UCN3 protein comprises immunostaining.

22. The method of claim 1 wherein assaying the β-cell or population of β-cells for elevated levels of UCN3 mRNA comprises conducting one or more hybridization assays.

23. The method of claim 22 wherein the one or more hybridization assays comprises a microarray.

24. The method of claim 1 wherein the presence of elevated levels of UCN3 mRNA comprises at least a 5 fold increase in the levels of UCN3 mRNA expression in the mature β-cells relative to the levels of UCN3 mRNA expression in the immature β-cells.

25. The method of claim 1 further comprising sorting the functionally immature and mature β-cells identified in the population of β-cells.

26. The method of claim 25 wherein sorting the functionally immature and mature β-cells identified in the population of β-cells comprises fluorescence-activated cell sorting (FACS).

27. The method of claim 25 further comprising quantifying the sorted functionally immature and mature β-cells identified in the population of β-cells.

28. The method of claim 25 further comprising preserving the sorted functionally mature β-cells.

29. A method of identifying an agent that modulates the functional maturity of β-cells, comprising:
(a) contacting β-cells or β-like cells with a test agent;
(b) assaying the cells contacted with the test agent for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and
(c) identifying the test agent as a candidate agent that modulates the functional maturity of β-cells, wherein:
(i) the test agent is a candidate agent that induces β-cells to become functionally immature if the β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNa; or
(ii) the test agent is a candidate agent that induces β-cells to become functionally mature if the β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

30-33. (canceled)

34. A method of identifying the functional maturity of an individual's β-cells, comprising:
(a) obtaining a biological sample comprising β-cells from the individual; and
(b) assaying the β-cells in the biological sample for the presence or absence of one or more of a GSIS response at low glucose concentrations, a large fold change in the GSIS response between low and high glucose concentrations, urocortin 3 (UCN3) protein, and elevated levels of UCN3 messenger ribonucleic acid (mRNA); and
(c) identifying the functional maturity of the individual's β-cells, wherein the individual's β-cells are:
(i) functionally immature if the individual's β-cells exhibit one or more of the presence of a GSIS response at low glucose concentrations, the absence of the large fold change in the GSIS response between low and high glucose concentrations, the absence of UCN3 protein, or the absence of elevated levels of UCN3 mRNa; or
(ii) functionally mature if the individual's β-cells exhibit one or more of the absence of a GSIS response at low glucose concentrations, the presence of the large fold change in the GSIS response between low and high glucose concentrations, the presence of UCN3 protein, or the presence of elevated levels of UCN3 mRNA.

35-38. (canceled)
